SEQUENCE LISTING (14 sequences)

SEQ ID NO: 1 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 2 - DNA, 2814 bp, Homo sapiens
atgcaccggccgcgccgccgcgggacgcgcccgccgctcctggcgctgctggccgcgctgctgctggccgcacgcggggctgctgcccaagaaacagagctgtcagtcagtgctgaattagtgcctacctcatcatggaacatctcaagtgaactcaacaaagattcttacctgaccctcgatgaaccaatgaataacatcaccacgtctctgggccagacagcagaactgcactgcaaagtctctgggaatccacctcccaccatccgctggttcaaaaatgatgctcctgtggtccaggagccccggaggctctcctttcggtccaccatctatggctctcggctgcggattagaaacctcgacaccacagacacaggctacttccagtgcgtggcaacaaacggcaaggaggtggtttcttccactggagtcttgtttgtcaagtttggcccccctcccactgcaagtccaggatactcagatgagtatgaagaagatggattctgtcagccatacagagggattgcatgtgcaagatttattggcaaccgcaccgtctatatggagtctttgcacatgcaaggggaaatagaaaatcagatcacagctgccttcactatgattggcacttccagtcacttatctgataagtgttctcagttcgccattccttccctgtgccactatgccttcccgtactgcgatgaaacttcatccgtcccaaagccccgtgacttgtgtcgcgatgaatgtgaaatcctggagaatgtcctgtgtcaaacagagtacatttttgcaagatcaaatcccatgattctgatgaggctgaaactgccaaactgtgaagatctcccccagccagagagcccagaagctgcgaactgtatccggattggaattcccatggcagatcctataaataaaaatcacaagtgttataacagcacaggtgtggactaccgggggaccgtcagtgtgaccaaatcagggcgccagtgccagccatggaattcccagtatccccacacacacactttcaccgcccttcgtttcccagagctgaatggaggccattcctactgccgcaacccagggaatcaaaaggaagctccctggtgcttcaccttggatgaaaactttaagtctgatctgtgtgacatcccagcgtgcgattcaaaggattccaaggagaagaataaaatggaaatcctgtacatactagtgccaagtgtggccattcccctggccattgctttactcttcttcttcatttgcgtctgtcggaataaccagaagtcatcgtcggcaccagtccagaggcaaccaaaacacgtcagaggtcaaaatgtagagatgtcaatgctgaatgcatataaacccaagagcaaggctaaagagctacctctttctgctgtacgctttatggaagaattgggtgagtgtgcctttggaaaaatctataaaggccatctctatctcccaggcatggaccatgctcagctggttgctatcaagaccttgaaagactataacaacccccagcaatggacggaatttcaacaagaagcctccctaatggcagaactgcaccaccccaatattgtctgccttctaggtgccgtcactcaggaacaacctgtgtgcatgctttttgagtatattaatcagggggatctccatgagttcctcatcatgagatccccacactctgatgttggctgcagcagtgatgaagatgggactgtgaaatccagcctggaccacggagattttctgcacattgcaattcagattgcagctggcatggaatacctgtctagtcacttctttgtccacaaggaccttgcagctcgcaatattttaatcggagagcaacttcatgtaaagatttcagacttggggctttccagagaaatttactccgctgattactacagggtccagagtaagtccttgctgcccattcgctggatgccccctgaagccatcatgtatggcaaattctcttctgattcagatatctggtcctttggggttgtcttgtgggagattttcagttttggactccagccatattatggattcagtaaccaggaagtgattgagatggtgagaaaacggcagctcttaccatgctctgaagactgcccacccagaatgtacagcctcatgacagagtgctggaatgagattccttctaggagaccaagatttaaagatattcacgtccggcttcggtcctgggagggactctcaagtcacacaagctctactactccttcagggggaaatgccaccacacagacaacctccctcagtgccagcccagtgagtaatctcagtaaccccagatatcctaattacatgttcccgagccagggtattacaccacagggccagattgctggtttcattggcccgccaatacctcagaaccagcgattcattcccatcaatggatacccaatacctcctggatatgcagcgtttccagctgcccactaccagccaacaggtcctcccagagtgattcagcactgcccacctcccaagagtcggtccccaagcagtgccagtgggtcgactagcactggccatgtgactagcttgccctcatcaggatccaatcaggaagcaaatattcctttactaccacacatgtcaattccaaatcatcctggtggaatgggtatcaccgtttttggcaacaaatctcaaaaaccctacaaaattgactcaaagcaagcatctttactaggagacgccaatattcatggacacaccgaatctatgatttctgcagaactgtaa

SEQ ID NO: 3 - DNA, 22 bp, Homo sapiens
accgcaccgtctatatggagtc

SEQ ID NO: 4 - DNA, 21 bp, Homo sapiens
gcatagtggcacacgggaagg

SEQ ID NO: 5 - DNA, 21 bp, Homo sapiens
accagcctcatgcacaaccac

SEQ ID NO: 6 - DNA, 20 bp, Homo sapiens
gcatgttgtccagccgcatc

SEQ ID NO: 7 - DNA, 21 bp, Homo sapiens
cctacggagcccaaggaactg

SEQ ID NO: 8 - DNA, 23 bp, Homo sapiens
cactctgtcatgttgcctgcact

SEQ ID NO: 9 - DNA, 22 bp, Homo sapiens
tgtccgaaggaatggaacacgg

SEQ ID NO: 10 - DNA, 25 bp, Homo sapiens
tttgagttcctctctctggtctccg

SEQ ID NO: 11 - DNA, 22 bp, Homo sapiens
tcctgcaccaccaactgcttag

SEQ ID NO: 12 - DNA, 22 bp, Homo sapiens
ggcatggactgtggtcatgagt

SEQ ID NO: 13 - DNA, 25 bp, Homo sapiens
cagaagcccattattcagagcgagt

SEQ ID NO: 14 - DNA, 20 bp, Homo sapiens
gcactctcgtcggtgactgt

(19) United States
(12) Patent Application Publication
(10) Pub. No.: US 2013/0131139 A1
(43) Pub. Date: May 23, 2013

(54) ROR1 AS A GENE TARGET IN ACUTE LYMPHOBLASTIC LEUKEMIA

(71) Applicants: Jeffrey W. Tyner, Portland, OR (US); Brian J. Druker, Portland, OR (US); Vincent Bicocca, Portland, OR (US); Bill H. Chang, Portland, OR (US)

(72) Inventors: Jeffrey W. Tyner, Portland, OR (US); Brian J. Druker, Portland, OR (US); Vincent Bicocca, Portland, OR (US); Bill H. Chang, Portland, OR (US)

(73) Assignee: Oregon Health & Science University

(21) Appl. No.: 13/299,245

(22) Filed: Nov. 17, 2011

(51) Int. Cl. A61K 31/713; A61K 31/7105; C40B 30/04; G01N 33/573; C12Q 1/48; C12Q 1/68; A61K 31/506; A61P 35/02

(52) U.S. Cl. 514/44 A; 514/252.19; 514/44 R; 435/7.4; 435/15; 435/6.13; 506/9; 435/6.12; 435/6.11; 435/6.14

(57) ABSTRACT

Disclosed are methods of selecting a subject suspected of having or having leukemia, such as lymphoblast leukemia (B-ALL), for treatment with an agent that inhibits ROR1-regulated signaling activity. In some examples, cells obtained from the subject are screened for over expression of ROR1. In other examples, the cells are contacted with an agent that inhibits ROR1 signaling activity and a ROR1-regulated signaling activity is detected. An alteration in the ROR1-regulated signaling activity as compared to a control identifies the subject as one that would benefit from treatment with an agent that inhibits ROR1 signaling activity. Also disclosed are methods for identifying an agent for treating a subject with a ROR1-dependent leukemia or with a predisposition for developing a ROR1-dependent leukemia, and methods for treating or inhibiting a ROR1-dependent leukemia, such as B-ALL in a subject.

### ACKNOWLEDGMENT OF GOVERNMENT SUPPORT

This invention was made with government support under grant number UL1 RR024140 awarded by the National Cancer Institute, and the Oregon Clinical and Translational Research Institute (OCTRI) from the National Center for Research Resources (NRCC), a component of the NIH. The government has certain rights in the invention.

### FIELD

This disclosure relates to the field of leukemia and in particular, to methods for detecting and treating leukemia targeting the receptor tyrosine kinase ROR1.

### BACKGROUND

The likelihood that a young person will develop cancer before adulthood is 1 in 300. As such, childhood cancer is the leading cause of disease-related mortality among children 1 to 14 years of age, and acute lymphoblastic leukemia (ALL) is a common form of childhood malignancy, accounting for approximately 25% of childhood cancers. Although great strides have been made in the treatment of childhood leukemias, such as ALL, one-third of patients will eventually succumb to the disease. To improve outcomes for these patients, new therapeutic strategies need to be developed that specifically target the cellular processes causing malignancy. This will require a more detailed understanding of the genetic lesions contributing to oncogenesis in each patient. The tyrosine kinase gene family has previously been implicated in the pathogenesis of numerous types of cancer.

Tyrosine kinases have critical roles in malignant transformation and are mutated or aberrantly expressed in multiple malignancies. A groundbreaking advance came with the identification of the t(9;22) chromosomal abnormality that generates the constitutively active fusion tyrosine kinase, BCR-ABL. This translocation has been identified as the causative transforming event in chronic myeloid leukemia (CML) and is also found in 20% of adult and 5% of pediatric acute lymphoblastic leukemia (ALL). Selective inhibitors of tyrosine kinases, such as imatinib, are now being used to treat the BCR-ABL positive subset of ALL patients with great success.

Recent studies have also shown the importance of tyrosine kinases in some subsets of pediatric ALL. For example, pediatric ALL patients carrying the hyperdiploid karyotype (>50 chromosomes) and infant ALL patients (<1 year of age) with ALL gene rearrangement have both been shown to either overexpress or carry activating mutations of the kinase, FLT3. Therefore, targeting FLT3 may improve the treatment of these specific ALL subtypes. In addition, activating mutations in the JAK family of tyrosine kinases and gene fusions of the upstream receptor, CRLF2, have been identified in approximately 5% of pediatric pre-B ALL patients with an enrichment of these lesions observed in patients with Down syndrome. For these patients, application of JAK kinase inhibitors may improve outcomes. However, with these few exceptions, specific kinase targets for therapeutic intervention in ALL have yet to be elucidated. Thus, the need exists for the identification of additional kinase targets amenable to targeted therapy.

### SUMMARY

Methods are disclosed for selecting a subject having or suspected of having leukemia, for example lymphoblastic leukemia (ALL), for treatment with an agent that inhibits ROR1-regulated signaling activity. In some examples, a subject suspected of having or having a leukemia is selected. In some examples, cells obtained from the subject are screened for over expression of ROR1. Over expression of ROR1 relative to a control identifies the subject as one that would benefit from treatment with an agent that inhibits ROR1, BTK and/or LYN signaling activity. In some examples, white blood cells or bone marrow cells obtained from the subject are contacted with an agent that inhibits ROR1 signaling activity and a ROR1-regulated signaling activity is detected, such as one or more of phosphorylation, cell viability, or cell proliferation. An alteration in the ROR1-regulated signaling activity in the white blood cells or bone marrow cells as compared to a control identifies the subject as one that would benefit from treatment with an agent that inhibits ROR1, BTK and/or LYN signaling activity. In some examples, the ROR1-regulated signaling activity is one or both of cell proliferation and cell viability of the white blood cells or bone marrow cells, and a decrease in one or both of cell proliferation and cell viability of the white blood cells or bone marrow cells as compared to the control identifies the subject as one that would benefit from treatment with an agent that inhibits ROR1 signaling activity. In some examples, the ROR1-regulated signaling activity is the phosphorylation of components in the biological signaling pathway including ROR1, and a decrease in phosphorylation of a downstream target in the biological signaling pathway including ROR1 identifies the subject as one that would benefit from treatment with an agent that inhibits ROR1, BTK and/or LYN signaling activity. In some examples, the ROR1-regulated signaling activity is the phosphorylation of components in the biological signaling pathway including ROR1, and an increase in phosphorylation of a downstream target in the biological signaling pathway including ROR1 identifies the subject as one that would benefit from treatment with an agent that inhibits ROR1, BTK and/or LYN signaling activity.

Also disclosed is a method for identifying an agent of use in treating a subject with a ROR1-dependent leukemia or with a predisposition for developing a ROR1-dependent leukemia. In such methods an isolated test cell that is dependent on ROR1 signaling activity is contacted with a test agent and a ROR1-regulated signaling activity is detected. The amount of the ROR1-regulated signaling activity in the test cell is compared to a control, wherein an alteration in the ROR1-regulated signaling activity in the test cell relative to the control indicates that the agent is useful for the treatment of a subject with ROR1-dependent leukemia or with a predisposition for developing a ROR1-dependent leukemia. In some examples, the ROR1-regulated signaling activity is one or both of cell proliferation and cell viability of the test cell, and a decrease in one or both of cell proliferation and cell viability of test cell as compared to a control identifies the agent as useful for the treatment of a subject with ROR1-dependent leukemia or with a predisposition for developing a ROR1-dependent leukemia. In some examples, the ROR1-regulated signaling activity is the phosphorylation of components in the biological signaling pathway including ROR1, and a decrease in phosphorylation of a downstream target in the biological signaling pathway including ROR1 identifies the agent as useful for the treatment of a subject with ROR1-dependent leukemia or with a predisposition for developing a ROR1-dependent leukemia. In some examples, the ROR1-regulated signaling activity is the phosphorylation of components in the biological signaling pathway that includes ROR1, and an increase in phosphorylation of a downstream target in the biological signaling pathway that includes ROR1 identifies the agent as useful for the treatment of a subject with ROR1-dependent leukemia or with a predisposition for developing a ROR1-dependent leukemia.

Also disclosed is a method for treating or inhibiting a ROR1-dependent leukemia, such as ALL in a subject. In some examples, a subject with ROR1-dependent leukemia is selected. In some examples, the subject is administered an effective amount of an inhibitor of ROR1, BTK and/or LYN signaling activity, such that the ROR1-dependent leukemia in the subject is treated.

The foregoing and other features and advantages of this disclosure will become more apparent from the following detailed description, which proceeds with reference to the accompanying figures.

### BRIEF DESCRIPTION OF THE DRAWINGS

FIGS. 1A-1G are graphs and digital images of a gel and a Western blot, showing that ROR1 is a functional target and uniformly expressed in t(1;19) ALL. FIG. A; white Blood cells (2.25×10⁷) from a t(1;19)-positive ALL patient (07-112) were suspended in siPORT® buffer and incubated with 1 μM siRNA from an siRNA library individually targeting each member of the tyrosine kinase family as well as N-RAS, K-RAS, and single and pooled non-specific siRNA controls. Cells were electroporated on a 96-well electroporation plate at 1110 V (equivalent of 150 V), 200 μsec, 2 pulses. Cells were replated into culture media and cell viability was determined by addition of a tetrazolium salt (MTS assay) at day 4 post-electroporation. Values represent percent mean (normalized to the median value on the plate)±standard error in the mean (s.e.m.) (n=3). FIG. 1B; White Blood cells (2.25×10⁷) from a normal karyotype (t(1;19)-negative) ALL patient (08-026) were treated as in FIG. 1A. Values represent percent mean (normalized to untreated controls)±s.e.m. (n=3). p<0.05. FIG. 1C; cDNA derived from white blood cells from patients 07-112 and 08-026 (FIGS. 1A and 1B) as well as from Ba/F3 parental cells or those ectopically expressing human ROR1 was amplified using ROR1-specific primers and PCR products were analyzed by gel electrophoresis. FIG. 1D; cDNA derived from t(1;19)-positive and negative cell lines and primary patient samples was amplified using primers specific for ROR1, E2A-PBX1, or GAPDH and PCR products were analyzed by gel electrophoresis. FIG. 1E; whole cell extracts derived from t(1;19)-positive and negative cell lines and primary patient samples was subjected to immunoblot analysis using antibodies specific for ROR1, E2A-PBX1, or β-ACTIN. FIG. 1F; flow cytometric analysis of t(1;19)-positive and negative cells lines was performed using specific polyclonal anti-human ROR1 antibodies (dark grey histogram) versus isotype control (light grey histogram). FIG. 1G, gene expression microarray data for pediatric ALL patients and normal B-cell progenitor populations were compiled into a meta-analysis. Patients with MLL gene rearrangements, t(9;22) (BCR-ABL), t(12;21) (TEL-AML), or t(1;19) (E2A-PBX1) (n=15 for each subset) and non-malignant B-cell progenitor populations (CD34+ Lin−, pro-B, pre-B, pre-BII large, pre-BII small, and immature B) (n=15 total) were examined and Affymetrix intensity values for ROR1 are plotted for each individual patient sample.

FIGS. 2A-2D are graphs and digital images of a Western blot and a gel showing that ROR1 (WT) is a therapeutic target candidate in t(1;19)-positive ALL. FIG. 2A; RCH-ACV or Kasumi-2 cells (both t(1;19)-positive) as well as REH cells (t(12;21)-positive) were electroporated in the presence of non-specific siRNA or siRNA targeting ROR1 and plated into culture media. After 4 days, cells were subjected to an MTS assay to measure cell viability. Values represent percent mean (normalized to non-specific control wells)±s.e.m. (n=10). FIG. 2B; RCH-ACV cells were electroporated in the presence of an individual duplex of non-specific siRNA or 3 individual duplexes of siRNA targeting different portions of ROR1 and cells were plated into culture media. After 3 days, cells were lysed and subjected to immunoblot with antibodies specific for ROR1 or β-ACTIN. After 4 days, parallel cultures of cells were subjected to an MTS assay to measure cell viability. Values represent percent mean (normalized to non-specific control wells)±s.e.m. (n=6). FIG. 2C; primary cells from a t(1;19) ALL patient were propagated in NOD-SCID mice lacking the IL-2 receptor γ chain. Xenograft cells were harvested from bone marrow and spleen of overtly leukemic mice and electroporated with non-specific or ROR1-targeting siRNA. After 4 days, cells were subjected to an MTS assay to measure cell viability. Values represent percent mean (normalized to non-specific control wells)±s.e.m. (n=4). FIG. 2D; primary cells from a t(1;19) ALL patient were propagated in a xenograft mouse model as above and RNA was harvested from cell extracts. PCR was performed on cDNA with primers specific for ROR1, E2A-PBX1, or GAPDH. The t(1;19) positive (RCH-ACV) and negative (REH) cell lines were included for comparison.

FIGS. 3A-3E are graphs and a digital image of a gel showing that dasatinib impairs t(1;19) cell growth by inhibiting pre-BCR signaling. FIG. 3A; RCH-ACV, Kasumi-2, and REH cells were cultured in graded concentrations of the kinase inhibitor, dasatinib, for 3 days at which time cells were subjected to an MTS assay for measurement of cell viability. Values represent percent mean (normalized to no-drug control wells)±s.e.m. (n=6). FIG. 3B; malignant cells from 10 pediatric ALL patients exhibiting a variety of chromosomal translocations (2 positive for t(1;19)) were cultured in graded concentrations of the kinase inhibitor, dasatinib, for 3 days at which time cells were subjected to an MTS assay for measurement of cell viability. Values represent percent mean (normalized to no-drug control wells)±s.e.m. (n=3). FIG. 3C; cDNA derived from primary samples of t(1;19) ALL patients 09-809 and 09-812 was amplified using primers specific for ROR1, E2A-PBX1, or GAPDH. The t(1;19) positive (RCH-ACV) and negative (REH) cell lines were included for comparison. FIG. 3D; RCH-ACV cells were cultured in graded concentrations of dasatinib for 96 hours before being stained with Annexin V-PE and 7-AAD and analyzed by flow cytometry to determine induction of apoptosis. Values represent the percentage of total positive cells±s.e.m. (n=3). FIG. 3E; RCH-ACV cells were electroporated in the presence of non-specific siRNA or siRNA targeting BTK, LYN or both BTK and LYN and then plated into culture media. After 4 days, cells were subjected to an MTS assay to measure cell viability. Values represent percent mean (normalized to non-specific control wells)±s.e.m. (n=5).

FIGS. 4A-4D are graphs and a digital image of a Western blot showing that inhibition of pre-BCR signaling by dasatinib induces ROR1 upregulation. FIG. 4A; RCH-ACV cells were cultured in the presence of 100 nM dasatinib for 12, 24, and 48 hours before RNA was harvested from cell extracts. Quantitative PCR was performed on cDNA with primers specific for ROR1 and GAPDH to determine relative ROR1 mRNA expression levels. Values represent the fold change (normalized to untreated control cells)±s.e.m. (n=6). FIG. 4B; RCH-ACV cells were cultured in the presence of 100 nM dasatinib for 12, 24, and 48 hours before cells were lysed and subjected to immunoblot analyses with antibodies specific for ROR1 or β-ACTIN to determine relative ROR1 protein expression. FIG. 4C; RCH-ACV cells were cultured in the presence of 100 nM dasatinib for 12, 24, 48 and 72 hours before cells were analyzed by flow cytometry using antibodies specific for ROR1 to determine relative ROR1 protein surface expression. Values represent mean fluorescent intensity as a percent of untreated±s.e.m. (n=4). FIG. 4D; RCH-ACV cells were electroporated in the presence of non-specific siRNA or siRNA targeting both BTK and LYN and then plated in culture media. After 3 days, RNA was harvested from cell extracts and quantitative PCR was performed on cDNA using primers specific for ROR1 and GAPDH to determine relative ROR1 mRNA expression levels. Values represent the percent change (normalized non-specific control cells)±s.e.m. (n=4).

FIGS. 5A-5F are graphs plots and a schematic showing ROR1 dependence and dasatinib sensitivity in B-cell malignancies FIG. 5A; RCH-ACV cells were electroporated in the presence of non-specific siRNA or siRNA targeting ROR1 and then plated in culture media. After 2 days, graded concentrations of dasatinib were added. Cells were allowed to culture an additional 2 days before they were subjected to an MTS assay for measurement of cell viability. Values represent percent mean (normalized to no-drug control wells)±s.e.m. (n=6). FIG. 5B; Primary cells from ALL patients with t(12;21), MLL rearrangement, t(1;19), and t(17;19) were analyzed by flow cytometry. Leukemic blasts were gated based on CD45 and side scatter and these blasts were evaluated for CD34 expression. FIG. 5C; cDNA derived from t(1;19) ALL, t(17;19) ALL, Burkitt's lymphoma, and normal karyotype ALL primary samples was analyzed for relative μHC mRNA expression using quantitative PCR. cDNA was amplified using primers specific for μHC and GUSB, and μHC levels were normalized to GUSB. FIG. 5D; cDNA derived from t(1;19) ALL, t(17;19) ALL, Burkitt's lymphoma, and normal karyotype ALL primary samples was analyzed for relative ROR1 mRNA expression using quantitative PCR. cDNA was amplified using primers specific for ROR1 and GAPDH, and ROR1 levels were normalized to GAPDH. FIG. 5E; t(1;19) ALL, t(17;19) ALL, Burkitt's lymphoma, and normal karyotype primary samples were cultured in the presence of graded concentrations of dasatinib. After 3 days, cells were subjected to an MTS assay for measurement of cell viability. Values represent percent mean, normalized to no-drug control wells. FIG. 5F; Normal B-cell progenitors naturally upregulate expression of ROR1 as well as the pre-BCR complex at the pre-BII stage of development. The t(1;19) oncogenic lesion generates the E2A-PBX1 chimeric transcription factor that contributes to the developmental arrest of B-cell progenitors at the pre-BII stage. Expression of ROR1 and signaling from the pre-BCR complex remains critical for viability of malignant cells that are arrested at this stage. Although ROR1 and the pre-BCR complex are not mutated oncogenes, they do represent onco-requisite pathways and so remain viable therapeutic targets for the treatment of t(1;19).

FIG. 6 is a Table showing primer sequences used for ROR1, WNT16B, E2A-PBX1, μHC, GAPDH, and GUSB.

FIG. 7 is a graph showing that the loss of E2A-PBX1 has no effect on ROR1 expression. siRNA mediated depletion of the E2A-PBX1 chimeric transcription factor in RCH-ACV cells result in a concomitant loss of the E2A-PBX1 transcriptional target WNT16B. ROR1 expression, however, is unaffected by loss of E2A-PBX1.

FIG. 8 is a set of graphs showing ROR1 loss impairs t(1;19) ALL cell growth and increases apoptosis. RCH-ACV cells electroporated with non-specific control siRNA or ROR1-specific siRNA were grown in culture for 96 hours before being counted and stained for the apoptosis marker ANNEXIN-V. Due to electroporation conditions, doubling rates are slower and baseline ANNEXIN-V positivity is higher in control non-specific siRNA treated cells compared to unelectroporated RCH-ACV control cultures. Still, cells treated with ROR1-specific siRNA show significantly impaired cellular outgrowth (left panel) and elevated ANNEXIN-V staining (right panel).

FIG. 9 is a digital image of a Western blot showing that ROR1 shows no evidence of tyrosine phosphorylation in RCH-ACV cells. Whole cell extracts from RCH-ACV cells were incubated with ROR1 antibody, which was precipitated with Protein G agarose beads. The immunoprecipitate was eluted from beads in sample buffer and divided into two equal parts. The immunoprecipitates, as well as RCH-ACV whole cell extract (WCE) and ROR1 antibody-depleted whole-cell extract flow through (WCE FT), were subjected to SDS-PAGE followed by immunoblot analysis using antibodies specific for ROR1 or phospho-tyrosine (4G10).

FIG. 10 is a graph shoeing μ heavy chain (μHC) expression in B-cell ALL and normal B-cell development. A metaanalysis of gene expression microarray data generated from pediatric ALL patient samples and normal B-cell progenitor populations revealed significantly higher average μHC expression levels in E2A-PBX1-positive ALL compared to other common translocations observed in pediatric ALL (BCR-ABL, TEL-AML, MLL rearrangements). The elevated μHC expression in E2A-PBX1-positive samples correlates with the elevated expression levels observed in intermediate B-cell progenitor populations (pre-BI, pre-BII large/small).

FIG. 11A-11B is a set of digital images of Western blots showing dasatinib treatment inhibits pre-BCR signaling in t(1;19) ALL. Downstream effectors and targets of pre-BCR signaling were analyses to determine the effect of dasatinib on pre-BCR activity. (A) AKT phosphorylation is rapidly inhibited following dasatinib treatment in RCH-ACV cells. Inhibition of AKT phosphorylation by dasatinib supports the hypothesis that dasatinib inhibits pre-BCR signaling, as PI3K is a known downstream effector of pre-BCR signaling, while dasatinib has no direct activity against PI3K or AKT. Similarly, the transcription repressor BCL6 is known to be upregulated in t(1;19) ALL due constitutive signaling through the pre-BCR. (B) Treatment of RCH-ACV cells with dasatinib results in loss of BCL6 protein expression.

### SEQUENCE LISTING

The nucleic and amino acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO: 1 is an exemplary amino acid sequence of human ROR1.

SEQ ID NO: 2 is an exemplary nucleic acid sequence of human ROR1.

SEQ ID NOs: 3-14 are the nucleic acid sequences of primers.

The Sequence Listing is submitted as an ASCII text file in the form of the file named Sequence.txt, which was created on Nov. 14, 2011, and is 13,857 bytes, which is incorporated by reference herein.

### DETAILED DESCRIPTION

### I. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). Terms describing protein structure and structural elements of proteins can be found in Creighton, Proteins, Structures and Molecular Properties, W.H. Freeman & Co., New York, 1993 (ISBN 0-717-7030).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of terms are provided:

Aberrant signaling activity of a protein: Inappropriate or uncontrolled signaling activity by protein, such as ROR1, for example by over-expression, upstream activation and/or mutation (for example a truncation, deletion, insertion and/translocation which increases the signaling activity), which can lead to uncontrolled cell growth and/or proliferation, for example in cancer, such as leukemia, for example ALL, such as a B-cell ALL (B-ALL). Thus, in some examples, the aberrant signaling activity of a protein includes and/or leads to uncontrolled cell growth and/or proliferation. In other examples, aberrant signaling activity of a protein includes an increase or decrease in kinase activity of a tyrosine kinase, which can lead to uncontrolled cell growth and/or proliferation. In still other examples, aberrant signaling activity of a protein includes an increase or decrease in a protein-protein interaction (for example, a protein-protein interaction involving ROR1), such as formation of a protein signaling complex. Additional examples of aberrant signaling activity of a protein include, but are not limited to, mislocalization of the protein, for example mislocalization in an organelle of a cell or mislocalization at the cell membrane. In some examples, the “aberrant signaling activity” of ROR1 refers to an increase in ROR1 signaling activity as measured by direct determination of ROR1 activity, or by an upstream regulator of ROR1 activity, or downstream effect of ROR1 activity, such as increased cell growth or proliferation. In some examples, signaling activity include kinase activity and aberrant signaling activity includes aberrant kinase activity as described herein.

Administration: The introduction of a composition into a subject by a chosen route. Administration can be local or systemic. For example, if the chosen route is intravenous, the composition is administered by introducing the composition into a vein of the subject.

Amplification: To increase the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample, for example the number of a ROR1 nucleic acid. An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

A commonly used method for real-time quantitative polymerase chain reaction involves the use of a double stranded DNA dye (such as SYBR Green I dye). For example, as the amount of PCR product increases, more SYBR Green I dye binds to DNA, resulting in a steady increase in fluorescence. Another commonly used method is real-time quantitative TAQMAN® PCR (Applied Biosystems). This type of PCR has reduced the variability traditionally associated with quantitative PCR, thus allowing the routine and reliable quantification of PCR products to produce sensitive, accurate, and reproducible measurements of levels of gene expression. The 5′ nuclease assay provides a real-time method for detecting only specific amplification products. During amplification, annealing of the probe to its target sequence generates a substrate that is cleaved by the 5′ nuclease activity of Taq DNA polymerase when the enzyme extends from an upstream primer into the region of the probe. This dependence on polymerization ensures that cleavage of the probe occurs only if the target sequence is being amplified. The use of fluorogenic probes makes it possible to eliminate post-PCR processing for the analysis of probe degradation. The probe is an oligonucleotide with both a reporter fluorescent dye and a quencher dye attached. While the probe is intact, the proximity of the quencher greatly reduces the fluorescence emitted by the reporter dye by Förster resonance energy transfer (FRET) through space. Probe design and synthesis has been simplified by the finding that adequate quenching is observed for probes with the reporter at the 5′ end and the quencher at the 3′ end.

Animal: A living multicellular vertebrate organism, a category that includes, for example, mammals and birds. A “mammal” includes both human and non-human mammals, such as mice. The term “subject” includes both human and animal subjects, such as mice. In some examples, a subject is a patient, such as a patient diagnosed with ALL, such as a pediatric patient diagnosed with ALL, for example B-ALL, such as t(1;19) B-ALL. Thus, the terms patient or subject can be used to refer to one diagnosed with ALL, such as pediatric ALL, for example B-ALL.

Antibody: A polypeptide ligand including at least a light chain or heavy chain immunoglobulin variable region which specifically binds an epitope of an antigen, such as an epitope of ROR1 located on the exterior side of the cell membrane. The term “specifically binds” refers to, with respect to an antigen, the preferential association of an antibody or other ligand, in whole or part, with this polypeptide, such as ROR1. Examples of antibodies that specifically bind ROR1 are known in the art. A specific binding agent, such as an antibody binds substantially only to a defined target. It is recognized that a minor degree of non-specific interaction may occur between a molecule, such as a specific binding agent, and a non-target polypeptide. Nevertheless, specific binding can be distinguished as mediated through specific recognition of the antigen. Although selectively reactive antibodies bind antigen, they can do so with low affinity. Specific binding typically results in greater than 2-fold, such as greater than 5-fold, greater than 10-fold, or greater than 100-fold increase in amount of bound antibody or other ligand (per unit time) to a polypeptide, as compared to a non-target polypeptide.

A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Antibodies can be composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”), that specifically bind the antigen. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Anti-proliferative activity: An activity of a molecule, for example an inhibitory RNA, such as a siRNA, a small molecule and the like, which reduces proliferation of at least one cell type, but which may reduce the proliferation (either in absolute terms or in rate terms) of multiple different cell types (e.g., different cell lines, different species, etc.). In specific embodiments, anti-proliferative activity of an inhibitory RNA, such as a siRNA will be apparent against white blood cells or bone marrow cells obtained from a subject diagnosed with cancer, such as leukemia, for example ALL, such as a ROR1-dependent B-ALL.

Antisense compound: Refers to an oligomeric compound that is at least partially complementary to the region of a target nucleic acid molecule (such as a ROR1 gene product) to which it hybridizes. As used herein, an antisense compound that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression, for example a ROR1 target molecule.

Nonlimiting examples of antisense compounds include primers, probes, antisense oligonucleotides, siRNAs, miRNAs, shRNAs and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound. In particular examples, the antisense compound is an antisense oligonucleotide, siRNA or ribozyme.

In some embodiments an antisense molecule is designed to target (for example to repress the expression) ROR1, for example using the nucleic acid sequences of ROR1 set forth in the accompanying sequence listing.

Binding or stable binding: An association between two substances or molecules, such as the association of an antibody with a peptide (such as a ROR1 peptide), nucleic acid to another nucleic acid (such as the binding of a probe to ROR1 RNA or ROR1 cDNA), or the association of a protein with another protein or nucleic acid molecule. Binding can be detected by any procedure known to one skilled in the art, for example in the case of a nucleic acid encoding a ROR1 protein, such as by physical or functional properties of the target: oligonucleotide complex.

An oligonucleotide binds or stably binds to a target nucleic acid if a sufficient amount of the oligonucleotide forms base pairs or is hybridized to its target nucleic acid, for example the binding of an oligonucleotide, such as an siRNA to the nucleic acid sequence of ROR1 or the binding of a kinase inhibitor with a kinase, such as LYN. The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

Binding can be detected by either physical or functional properties. Binding between a target and an oligonucleotide can be detected by any procedure known to one skilled in the art, including both functional (for example reduction in expression and/or activity) and physical binding assays.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt. In another example, the method involves detecting a signal, such as a detectable label, present on one or both nucleic acid molecules (or antibody or protein as appropriate).

Biological signaling pathway: A systems of proteins, such as tyrosine kinases, and other molecules that act in an orchestrated fashion to mediate the response of a cell toward internal and external signals. In some examples, biological signaling pathways include tyrosine kinase proteins, which can propagate signals in the pathway by selectively phosphorylating downstream substrates. In some examples, a biological signaling pathway is disregulated and functions improperly, which can lead to aberrant signaling and in some instances hyper-proliferation of the cells with the aberrant signaling. In some examples, deregulation of a biological signaling pathway can result in a malignancy, such as cancer, for example a leukemia, such as B-ALL, for example a B-ALL malignancy characterized by an arrest in B-cell development at the intermediate/mature B-cell stage. A ROR1 biological signaling pathway, is a signaling pathway, in which ROR1 plays a roll, for example by receiving signals from upstream signaling molecules and/or phosphorylating downstream targets.

Biomarker: Molecular, biological or physical attributes that characterize a physiological state and can be objectively measured to detect or define disease progression or predict or quantify therapeutic responses. For instance, a substance used as an indicator of a biologic state, for example upregulation of expression of ROR1 is an indication of a B-cell malignancy in which the differentiation of the B-cells is arrested in an intermediate/mature B-cell state.

It is a characteristic that is objectively measured and evaluated as an indicator of normal biologic processes, pathogenic processes, or pharmacologic responses to a therapeutic intervention. In one example, a biomarker is a protein or nucleic acid sequence of a corresponding gene that is indicator of B-ALL, such as the expression of ROR1 proteins or nucleic acids.

Bruton tyrosine kinase (BTK): A non-receptor tyrosine kinase involved in for B lymphocyte development. Binding of antigen to the B-cell antigen receptor (BCR) triggers signaling leading to B-cell activation. BTK acts as a platform to bring together a diverse array of signaling proteins and is implicated in cytokine receptor signaling pathways. Representative examples of the protein sequence of BTK from Homo sapiens can be found on GENBANK® for example at gene accession no. NP_{—}000052, as available on Oct. 26, 2011, which is hereby incorporated reference in its entirety. Representative examples of the nucleic acid sequence of BTK from Homo sapiens can be found on GENBANK® for example at gene accession no. NM_{—}000061, as available on Oct. 26, 2011, which is hereby incorporated reference in its entirety.

A “BTK inhibitor” inhibits the signaling of a BTK protein, for example by inhibiting the kinase activity of BTK or the expression of BTK. Exemplary BTK inhibitors are siRNAs, ribozymes, antisense molecules, and small molecule kinase inhibitors, such as dasatinib.

Cancer: Malignant neoplasm that has undergone characteristic anaplasia with loss of differentiation, increase rate of growth, invasion of surrounding tissue, and is capable of metastasis.

Examples of hematological malignancies include leukemias, including acute leukemias (such as acute lymphocytic leukemia (ALL), acute myelocytic leukemia (AML) acute myelogenous leukemia and myeloblastic, promyelocytic, myelomonocytic (CMML), monocytic and erythroleukemia), chronic leukemias (such as chronic myelocytic (granulocytic) leukemia, chronic myelogenous leukemia (CML), chronic lymphocytic leukemia (CLL), chronic neutrophilic leukemia (CNL) and chronic myelomonocytic leukemia (CMML)), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, primary myelofibrosis, and myelodysplasia. Particular hematological malignancies associated with aberrant signaling activity are ALLs, such as B-ALLs, for example the ROR1-dependent B-ALLs described herein. In some specific examples, a B-ALL associated with ROR1 overexpression and/or dependence is a t(1;19) ALL, t(17;19) ALL and t(8;14)/Burkitt's ALL.

cDNA (complementary DNA): A piece of DNA lacking internal, non-coding segments (introns) and regulatory sequences which determine transcription. cDNA can be synthesized by reverse transcription from messenger RNA (mRNA) extracted from cells, for example ROR1 cDNA reverse transcribed from ROR1 mRNA. The amount of ROR1 cDNA reverse transcribed from ROR1 mRNA can be used to determine the amount of ROR1 mRNA present in a biological sample and thus the amount of expression of ROR1.

Cell proliferation: The ability of cells to multiply, for example through rounds of cell division. There are various methods of determining cell proliferation known to those of skill in the art and non-limiting examples of such methods are described in Section G.

Chemotherapy: In cancer treatment, chemotherapy refers to the administration of one or more agents (chemotherapeutic agents) to kill or slow the reproduction of rapidly multiplying cells, such as tumor or cancer cells. In a particular example, chemotherapy refers to the administration of one or more agents to significantly reduce the number of tumor cells in the subject, such as by at least about 50%. “Chemotherapeutic agents” include any chemical agent with therapeutic usefulness in the treatment of cancer. Chemotherapeutic agents include kinase inhibitors, such as inhibitors of the tyrosine kinases BTK and/or LYN.

Examples of chemotherapeutic agents can be found for example in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc.; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). A chemotherapeutic agent of use in a subject, such as a BTK and/or LYN kinase inhibitor, can decrease a sign or a symptom of a cancer, such as an ALL, for example B-ALL, or can reduce, stop or reverse the progression, metastasis and/or growth of a cancer.

Contacting: Placement in direct physical association including both in solid or liquid form. Contacting can occur in vivo, for example by administering an agent to a subject. “Administration” is the introduction of a composition, such as a composition containing an inhibitor of ROR1, BTK and/or LYN signaling activity, into a subject by a chosen route. For example, if the chosen route is intravenous, the composition is administered by introducing the composition into a vein of the subject. “Administrating” to a subject includes topical, parenteral, oral, intravenous, intra-muscular, sub-cutaneous, inhalational, nasal, or intra-articular administration, among others.

Control: A reference standard. A control can be a standard value a control cell not contacted with an agent. A control can be a known value indicative of basal expression of a gene, for example the amount of ROR1 expressed in peripheral blood cells, such as B-cells and/or lymphoid tissue in a subject that does not have B-ALL, such as a ROR1-dependent B-ALL. A difference between the expression in a test sample (such as a biological sample obtained from a subject) and a control can be an increase or conversely a decrease.

A difference between a test sample and a control can be an increase or conversely a decrease. The difference can be a qualitative difference or a quantitative difference, for example a statistically significant difference. In some examples, a difference is a decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Decreased expression, Downregulated, or Inactivation: When used in reference to the expression of a nucleic acid molecule, such as a gene, refers to any process which results in a decrease in production of a gene product. A gene product can be RNA (such as mRNA, rRNA, tRNA, and structural RNA) or protein. Therefore, gene downregulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a sample obtained from a subject who does not have B-ALL, or a standard value indicative of basal expression of a gene such as ROR1).

Degenerate variant: A polynucleotide encoding a protein of interest that includes a sequence that is degenerate as a result of the genetic code. For example, a polynucleotide encoding ROR1 includes a sequence that is degenerate as a result of the genetic code. There are 20 natural amino acids, most of which are specified by more than one codon. Therefore, all degenerate nucleotide sequences are included as long as the amino acid sequence of the ROR1 polypeptide encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Detect: To determine if an agent (such as a signal, particular nucleic acid, for example a ROR1 nucleic acid, or a protein, such as ROR1) is present or absent or the level of the agent that is present in a sample. In some examples, this can further include quantification, for example quantification of a ROR1 nucleic acid, or a ROR1 protein in a sample.

Determining expression, such as detecting expression of a gene product: Detection of a level of expression in either a qualitative or quantitative manner, for example by detecting nucleic acid or protein (such as a ROR1 nucleic acid or protein) by routine methods known in the art.

Diagnostic: Identifying the presence or nature of a pathologic condition, such as, but not limited to cancer, such as ALL, for example B-ALL. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (for example severity) of a pathologic condition, such as ALL, for example B-ALL.

Differential expression or altered expression: A difference, such as an increase or decrease, in the amount of messenger RNA, the conversion of mRNA to a protein, or both. In some examples, the difference is relative to a control or reference value, such as an amount of gene expression in tissue not affected by a disease, such as from a different subject who does not have B-ALL. Detecting differential expression can include measuring a change in gene or protein expression, such as a change in expression of ROR1

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes ROR1, or a fragment thereof, encompasses both the sense strand and its reverse complement.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

Laboratory standards and values may be set based on a known or determined population value (for example, a value representing expression of a gene for a particular parameter, such as expression of a gene that encodes ROR1) and can be supplied in the format of a graph or table that permits comparison of measured, experimentally determined values.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to a ROR1 encoding mRNA, a ROR1 encoding DNA, or a ROR1-encoding dsDNA.

“Specifically hybridizable” and “specifically complementary” are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

Increase Expression, Upregulated or Activation: When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 2-fold, for example at least 3-fold or at least 4-fold, as compared to a control (such an amount of gene expression in a sample obtained from a subject who does not have B-ALL disease, or a standard value indicative of basal expression of a gene, such as ROR1).

Inhibit: To reduce to a measurable extent for example, to reduce enzymatic activity. In some examples, the activity (such as signaling activity) of ROR1, BTK and/or LYN is inhibited, for example using a small molecule inhibitor of ROR1, BTK and/or LYN or an siRNA that inhibits the expression of ROR1, BTK and/or LYN.

Inhibiting or treating a disease: Inhibiting the full development of a disease or condition, for example, in a subject who is at risk for a disease such cancer, for example a leukemia, such as ALL, for example B-ALL. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and/organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids, such as probes and primers.

Kinase: An enzyme that catalyzes the transfer of a phosphate group from one molecule to another. Kinases play a role in the regulation of cell proliferation, differentiation, metabolism, migration, and survival. A “tyrosine kinase” transfers phosphate groups to a hydroxyl group of a tyrosine in a polypeptide. In some examples, a kinase is a LYN tyrosine kinase.

Receptor protein tyrosine kinases (PTKs), contain a single polypeptide chain with a transmembrane segment. The extracellular end of this segment contains a high affinity ligand-binding domain, while the cytoplasmic end comprises the catalytic core and the regulatory sequences.

Non-receptor tyrosine kinases, such as BTK and LYN, can be located in the cytoplasm as well as in the nucleus. They exhibit distinct kinase regulation, substrate phosphorylation, and function.

A “preferential” inhibition of a kinase refers to decreasing activity of one kinase, such as LYN more than inhibiting the activity of a second kinase, such as a mitogen-activated protein kinase (MAPK) or another tyrosine kinase.

Label: An agent capable of detection, for example by spectrophotometry, flow cytometry, or microscopy. For example, a label can be attached to a nucleotide, thereby permitting detection of the nucleotide, such as detection of the nucleic acid molecule of which the nucleotide is a part, such as a specific probe or primer. Labels can also be attached to antibodies, such as a ROR1 antibody. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Lymphocytes: A type of white blood cell that is involved in the immune defenses of the body. There are two main types of lymphocytes: B cells and T cells. T cells are white blood cells critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. B cells are white blood cells critical to the antibody response. B cells mature within the bone marrow and leave the marrow expressing an antigen binding antibody on their cell surface.

LYN: A member of the Src family of non-receptor protein tyrosine kinases. In various hematopoietic cells, Lyn has emerged as an enzyme involved in the regulation of cell activation. LYN is associated with cell surface receptor proteins, including the B cell antigen receptor (BCR). Representative examples of the protein sequence of LYN from Homo sapiens can be found on GENBANK® for example at gene accession no. NP_{—}002341 and NP_{—}001104567 as available on Oct. 26, 2011, which are hereby incorporated reference in their entirety. Representative examples of the nucleic acid sequence of LYN from Homo sapiens can be found on GENBANK® for example at gene accession no. NM_{—}001111097 and NM_{—}002350 as available on Oct. 26, 2011, which are hereby incorporated reference in their entirety.

A “LYN inhibitor” inhibits the signaling of a LYN protein, for example by inhibiting the kinase activity of LYN or the expression of LYN. Exemplary ROR1 inhibitors are antibodies that specifically bind LYN, siRNAs, ribozymes, antisense molecules, and small molecule kinase inhibitors, such as dasatinib.

Mass spectrometry: A method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (E1). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation. In some examples, ROR1 expression, and/or ROR1 over expression, is detected by mass spectrometry.

Nucleic acid molecules representing genes: Any nucleic acid, for example DNA (intron or exon or both), cDNA, or RNA (such as mRNA), of any length suitable for use as a probe or other indicator molecule, and that is informative about the corresponding gene.

Nucleic acid molecules: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, nucleic acid molecule can be circular or linear.

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides.

An oligonucleotide probe is a short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes.

Pharmaceutical composition: A chemical compound or composition capable of inducing a desired therapeutic or prophylactic effect when properly administered to a subject, for example a subject with B-ALL. A pharmaceutical composition can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject). In a particular example, a pharmaceutical agent is an agent that significantly reduces one or more symptoms associated with B-ALL.

Pharmaceutically acceptable carriers or vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as the treatments for B-ALL described herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In a particular embodiment the carrier is one that allows the therapeutic compound to cross the blood-brain barrier. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phospho-peptide or phospho-protein: A peptide or protein in which one or more phosphate moieties are covalently linked to amino acid residue, or amino acid analogs. A peptide can be phosphorylated at multiple or single sites. Sometimes it is desirable for the phospho-peptide to be phosphorylated at one site regardless of the presence of multiple potential phosphorylation sites. In vivo the transfer of a phosphate to a peptide is accomplished by a kinase exhibiting kinase activity, for example a tyrosine kinase, such as BTK or LYN transfers a phosphate to a tyrosine residue of a substrate peptide or protein.

Polymerizing agent: A compound capable of reacting monomer molecules (such as nucleotides) together in a chemical reaction to form linear chains or a three-dimensional network of polymer chains. A particular example of a polymerizing agent is polymerase, an enzyme, which catalyzes the 5′ to 3′ elongation of a primer strand complementary to a nucleic acid template. Examples of polymerases that can be used to amplify a nucleic acid molecule include, but are not limited to the E. coli DNA polymerase I, specifically the Klenow fragment which has 3′ to 5′ exonuclease activity, Taq polymerase, reverse transcriptase (such as HIV-1 RT), E. coli RNA polymerase, and wheat germ RNA polymerase II.

The choice of polymerase is dependent on the nucleic acid to be amplified. If the template is a single-stranded DNA molecule, a DNA-directed DNA or RNA polymerase can be used; if the template is a single-stranded RNA molecule, then a reverse transcriptase (such as an RNA-directed DNA polymerase) can be used. In some examples, a polymerizing agent is used to amplify a ROR1 nucleic acid, such as in a sample obtained from a subject, to detect a mutation in ROR1.

Polypeptide: Any chain of amino acids, regardless of length or post-translational modification (such as glycosylation, methylation, ubiquitination, phosphorylation, or the like). In one embodiment, a polypeptide is a ROR1 polypeptide. In one embodiment, a polypeptide is a BTK polypeptide. In one embodiment, a polypeptide is a LYN polypeptide. “Polypeptide” is used interchangeably with peptide or protein, and is used to refer to a polymer of amino acid residues. A “residue” refers to an amino acid or amino acid mimetic incorporated in a polypeptide by an amide bond or amide bond mimetic.

Probes and primers: A probe comprises an isolated nucleic acid capable of hybridizing to a target nucleic acid (such as a ROR1 nucleic acid molecule). A detectable label or reporter molecule can be attached to a probe. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998).

Probes are generally at least 12 nucleotides in length, such as at least 12, at least 13, at least 14, at least 15, at least 16, at least 17, at least 18, at least 19, least 20, at least 21, at least 22, at least 23, at least 24, at least 25, at least 26, at least 27, at least 28, at least 29, at least 30, or more contiguous nucleotides complementary to the target nucleic acid molecule, such as 12-30 nucleotides, 15-30 nucleotides, 20-30 nucleotides, or 12-29 nucleotides.

Primers are short nucleic acid molecules, for instance DNA oligonucleotides 10 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule (such as a portion of a ROR1 nucleic acid molecule).

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure (for example, to amplify a region of a ROR1 nucleic acid molecule) include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 15-50 nucleotides, 20-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

PCR primer pairs can be derived from a known sequence (such as the ROR1 nucleic acid molecule encoding the ROR1 amino acid sequence as set forth in SEQ ID NO: 2) for example, by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Prognosis: The probable course or outcome of a disease process. In several examples, the prognosis of a subject with cancer can indicate the likelihood of survival and/or the likelihood of metastasis. The prognosis of a subject with cancer, such as a leukemia, for example ALL (e.g. B-ALL), can indicate the likelihood that the subject will survive for a period of time, such as about one, about two, about three, about four, about five or about ten years. The prognosis of a subject with cancer can also indicate the likelihood of a cure, of the likelihood that the subject will remain disease-free following treatment for a period of time, such as about one, about two, about three, about four, about five or about ten years.

Quantitating: Determining or measuring a quantity (such as a relative quantity) of a molecule or the activity of a molecule, such as the quantity of ROR1 present in a sample.

RNA interference (RNAi): Refers to a cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. Double-stranded RNA molecules that inhibit gene expression through the RNAi pathway include siRNAs, miRNAs, and shRNAs.

ROR1: A protein also known as neurotrophic tyrosine kinase, receptor-related 1 (NTRKR1) encoded by the ROR1 gene. ROR1 is a member of the receptor tyrosine kinase-like orphan receptor (ROR) family. Representative examples of the protein sequence of ROR1 from Homo sapiens can be found on GENBANK® for example at gene accession no. NP_{—}005003, NP_{—}001077061, and Q01973 as available on Oct. 26, 2011, which are hereby incorporated reference in their entirety. Representative examples of the nucleic acid sequence of ROR1 from Homo sapiens can be found on GENBANK® for example at gene accession no. NM_{—}005012, NM_{—}001083592, and M97675 as available on Oct. 26, 2011, which are hereby incorporated reference in their entirety.

A “ROR1 inhibitor” inhibits the signaling activity of a ROR1 protein, for example by inhibiting phosphorylation events downstream of ROR1 or the expression of ROR1. Exemplary ROR1 inhibitors are antibodies that specifically bind ROR1, siRNAs, ribozymes, antisense molecules, and small molecule kinase inhibitors.

Sample: A sample, such as a biological sample, is a sample obtained from a plant or animal subject. As used herein, biological samples include all clinical samples useful for detection of the sensitivity of a subject to inhibitors of ROR1, BTK and/or LYN signaling activity, including, but not limited to, cells, tissues, and bodily fluids, such as: blood; derivatives and fractions of blood, tissue biopsy (such as a bone marrow biopsy, for example bone marrow cells), including tissues that are, for example, unfixed, frozen, fixed in formalin and/or embedded in paraffin, or bone marrow aspirates. In particular embodiments, the biological sample is obtained from a subject, such as in the form of blood or a fraction thereof such as leukocytes, lymphocytes, and/or mononuclear cells. In some examples, a sample is one obtained from a subject having, suspected of having, or who has had, for example diagnosed with a leukemia, such as ALL, for example B-ALL.

Screening: A process used to evaluate and identify candidate agents that decrease signaling activity of ROR1. In some cases, screening involves contacting a candidate agent (such as a small molecule, peptide or nucleic acid molecule) with cells that are dependent on the signaling activity of ROR1 and testing the effect of the agent on the viability of the cells and/or the signaling activity of ROR1.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Homologs and variants of a ROR1 protein are typically characterized by possession of at least 50% sequence identity counted over the full length alignment with the amino acid sequence of a native protein using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 60%, at least 65%, at least 70%, at least 75%, at least 80%, at least 90%, at least 95%, at least 96%, at least 97%, at least 98%, or at least 99% sequence identity. When less than the entire sequence is being compared for sequence identity, homologs and variants will typically possess at least 75% sequence identity over short windows of 10-20 amino acids, and may possess sequence identities of at least 85% or at least 90% at least 95%, at least 96%, at least 97%, at least 98%, or at least 99% depending on their similarity to the reference sequence. Methods for determining sequence identity over such short windows are described at the NCBI website. One of skill in the art will appreciate that these sequence identity ranges are provided for guidance only; it is entirely possible that strongly significant homologs could be obtained that fall outside of the ranges provided.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. One indication that two nucleic acid molecules are closely related is that the two molecules hybridize to each other under stringent conditions.

Short hairpin RNA (shRNA): A sequence of RNA that makes a tight hairpin turn and can be used to silence gene expression via the RNAi pathway. The shRNA hairpin structure is cleaved by the cellular machinery into siRNA.

Small interfering RNA (siRNA): A double-stranded nucleic acid molecule that modulates gene expression through the RNAi pathway. siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.”

Small molecule: A molecule, typically with a molecular weight less than about 1000 Daltons, or in some embodiments, less than about 500 Daltons, wherein the molecule is capable of modulating, to some measurable extent, a signaling activity of a target molecule such as inhibiting the signaling activity of ROR1 and/or inhibiting the signaling activity of a tyrosine kinase, such as the BTK and/or LYN tyrosine kinase.

Specific Binding Agent: An agent that binds substantially or preferentially only to a defined target such as a protein, enzyme, polysaccharide, oligonucleotide, DNA, RNA, recombinant vector or a small molecule. In an example, a “specific binding agent” is capable of binding to a ROR1 gene product, such as a ROR1 mRNA, cDNA, or protein. Thus, a nucleic acid-specific binding agent binds substantially only to the defined nucleic acid, such as RNA, or to a specific region within the nucleic acid.

A protein-specific binding agent binds substantially only the defined protein, or to a specific region within the protein. For example, a “specific binding agent” includes antibodies and other agents that bind substantially to a specified polypeptide, for example a specific binding agent that specifically binds ROR1, can be an antibody, for example a monoclonal or polyclonal antibody or a ligand for ROR1. Antibodies can be monoclonal or polyclonal antibodies that are specific for the polypeptide, such as ROR1, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Standard: A substance or solution of a substance of known amount, purity or concentration. A standard can be compared (such as by spectrometric, chromatographic, or spectrophotometric analysis) to an unknown sample (of the same or similar substance) to determine the presence of the substance in the sample and/or determine the amount, purity or concentration of the unknown sample. In one embodiment, a standard is a peptide standard. An internal standard is a compound that is added in a known amount to a sample prior to sample preparation and/or analysis and serves as a reference for calculating the concentrations of the components of the sample. In one example, nucleic acid standards serve as reference values for expression levels of specific nucleic acids, such as ROR1 nucleic acids. In some examples, peptide standards serve as reference values for expression levels of specific peptides, such as ROR1 proteins. Isotopically-labeled peptides are particularly useful as internal standards for peptide analysis since the chemical properties of the labeled peptide standards are almost identical to their non-labeled counterparts. Thus, during chemical sample preparation steps (such as chromatography, for example, HPLC) any loss of the non-labeled peptides is reflected in a similar loss of the labeled peptides.

Substrate: A molecule that is acted upon by an enzyme, such as ROR1. A substrate binds with the enzyme's active site, and an enzyme-substrate complex is formed. In some examples, the enzyme catalyses the incorporation of an atom or other molecule into the substrate, for example a kinase can incorporate a phosphate into the substrate, such as a peptide, thus forming a phospho-substrate.

Test agent: Any agent that is tested for its effects, for example its effects on the kinase activity of a kinase, such as the kinase activity of LYN or BTK, or the signaling activity of a protein, such as the signaling activity of ROR1, LYN and/or BTK. In some embodiments, a test agent is a chemical compound, such as a chemotherapeutic agent or even an agent with unknown biological properties.

Therapeutic agent: A chemical compound, small molecule, or other composition, such as an antisense compound, antibody, peptide, nucleic acid molecule, kinase inhibitor, capable of inducing a desired therapeutic or prophylactic effect when properly administered to a subject.

Therapeutically effective amount or Effective amount: The amount of agent, such as a chemotherapeutic agent, such as an inhibitor of ROR1 signaling activity, that is sufficient to prevent, treat (including prophylaxis), reduce and/or ameliorate the symptoms and/or underlying causes of any of a disorder or disease, for example to prevent, inhibit, and/or treat cancer, such as leukemia, for example ALL (e.g. B-ALL). In some embodiments, an “effective amount” is sufficient to reduce or eliminate a symptom of a disease, such as leukemia, for example ALL (e.g. B-ALL).

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

### II. Description of Several Embodiments

### A. Introduction

Acute lymphoblastic leukemia (ALL) is the most common form of pediatric cancer. With current treatment regimens many patients achieve relapse-free survival, however, patients with recurrent disease have a poor prognosis. Additionally, patients within certain subtypes of ALL still exhibit high risk for relapse, even with current standard of care treatment. Recently, addition of the drug imatinib to the therapeutic regimen of patients with a 9;22 chromosomal translocation, a subtype with historically poor prognosis, resulted in markedly improved outcomes for these patients. Imatinib specifically targets the cancer-causing gene fusion, BCR-ABL. These data suggest that addition of similar gene-targeted drugs to treatment regimens of patients from other ALL subtypes may improve outcomes. Unfortunately, the complete genetic etiology of disease for most patients remains unknown creating a dearth of gene targets for therapeutic intervention.

Many ALL patients can be categorized into distinct subsets of disease based on recurrent, somatic chromosomal translocations in their leukemic blasts. One of the most common, recurring translocations found in ALL patients is t(1;19)(q23;p13), which is observed in approximately 5% of all pediatric ALL cases as well as 1-2% of adult ALL cases. It is also rarely found in T-ALL and acute myeloid neoplasms. Greater than 90% of patients with t(1;19) exhibit blasts with expression of cytoplasmic Igμ and an absence of CD34 on the cell surface, indicating that these blasts are typically arrested at a later stage of B-cell differentiation (large/small pre-BII) than most other subsets of ALL patients. The 1;19 translocation occurs as a balanced translocation in 25% of patients, in which the reciprocal 19;1 chromosome is retained in the malignant cells, while the other 75% of patients exhibit a derivative chromosome 19 resulting in an unbalanced translocation. In both cases, the 1;19 translocation results in the fusion transcription factor complex, E2A-PBX1. The E2A-PBX1 oncogene has been shown to exhibit transforming capacity in fibroblasts in vitro, and can induce myeloid, T-lymphoid, and B-lymphoid malignancies in mouse models. On a molecular level, E2A-PBX1 has been shown to contribute to lymphoid neoplasia via enhanced expression of Bmi-1 leading to subsequent repression of the INK4A-ARF tumor suppressor locus. In addition, loss of E2A-PBX1 impairs t(1;19) ALL viability in part by disrupting the aberrant transcriptional activation of EB1 and WNT16B, thought to contribute to cellular transformation.

Clinically, patients with t(1;19) ALL have a higher probability of high risk disease by National Cancer Institute risk criteria. Further, the balanced t(1;19) karyotype was an adverse prognostic factor associated with elevated white blood cell counts and CNS involvement. However, intensified chemotherapy regimens have overcome the adverse prognostic significance of t(1;19) as an independent risk factor. Unfortunately, current intensive cytotoxic therapies for pediatric ALL are associated with long-term toxicities. Therefore, identification of new targets for therapy may lead to personalized treatments that can reduce the risk of these long-term toxicities and improve outcomes.

Although t(1;19) ALL patients generally exhibit 80-85% relapse free survival rates, many of these patients must tolerate intensive chemotherapy regimens to achieve remission. Further, there remains a subset of patients with t(1;19) who experience a relapse. Salvage therapies including stem cell transplant remain suboptimal with less than 50% retrieval. Therefore, these patients may benefit with the addition of therapies to improve outcomes. Additionally, standard cytotoxic drugs exhibit toxicities that can give rise to a multitude of long term side effects and secondary malignancies. As such, a gene targeted drug could offer great benefit to these patients by reducing rates of relapse and allowing patients to be treated with less intensive chemotherapy regimens while still exhibiting similar or more favorable outcomes.

As disclosed herein an RNAi-assisted protein target identification (RAPID) screen has identified a novel protein, ROR1, in t(1;19) ALL samples. It was found that all examined primary ALL samples and cell lines with this karyotype (both balanced and unbalanced translocations) overexpress ROR1, and that targeting of this gene reduces the viability of t(1;19) primary cells, xenograft cells, and cell lines. Thus, targeting of ROR1 expression and/or signaling activity may lead to improved strategies for treatment of t(1;19) ALL. Further, as disclosed herein primary t(1;19) ALL samples and cell lines have been identified that are sensitive to treatment with the FDA-approved small-molecule kinase inhibitor dasatinib due to inhibition of the tyrosine kinases BTK and LYN, which are critical components of the cytoplasmic pre-BCR signaling complex (expressed in over 90% of t(1;19) cases). Similarly, other subsets of patients with B-malignances that are arrested at intermediate or mature stages of B-cell developmental, such as an ALL patient with t(17;19) and a patient with Burkitts's (t(8;14)), also exhibit elevated expression of ROR1 and dasatinib sensitivity. Therefore, targeting BTK and LYN, with inhibitors of their signaling activity, such as with small molecule kinase inhibitors can be used for therapeutic intervention. Finally, as disclosed herein targeting ROR1 while simultaneously inhibiting the pre-BCR complex with dasatinib, provides an additive effect. Cumulatively, these findings indicate that therapies directed against ROR1 and the pre-BCR have potential benefit for patients with t(1;19) ALL as well as other B-lineage malignancies arrested at intermediate or mature stages of B-cell development.

### B. Method for Determining if a Subject is Susceptible to Treatment

Methods are provided herein for determining if a subject with a leukemia or suspected of having a leukemia, such as ALL, for example B-ALL and/or pediatric B-ALL, would benefit from treatment with an agent that inhibits the signaling activity of ROR1, BTK and/or LYN, for example to select a subject for treatment with an inhibitor of ROR1 signaling activity (for example a small molecule inhibitor of ROR1 signaling activity, an antibody that specifically binds the extracellular domain of ROR1 or a nucleic acid that inhibits expression of ROR1) or an inhibitor of BTK and/or LYN kinase activity (such as a small molecule inhibitor of BTK and/or LYN kinase activity, for example dasatinib). In some embodiments, the disclosed methods include selecting a subject diagnosed with leukemia such as B-ALL, for example B-ALL in which the stage of differentiation of the leukemic B-cells is arrested at the intermediate/mature stage of development, and determining if the subject will benefit from a therapy that reduces the signaling activity of ROR1, for example to treat the B-ALL. In some embodiments, a subject is selected that is suspected of having or having a t(1;19) B-ALL, a t(17;19) ALL or a t(8;14) Burkitt's ALL. In some embodiments, a biological sample obtained from the subject that includes leukemic cells (such as leukemic B-cells) to determine if the leukemic cells are ROR1 dependent, that is if the cells need the expression of ROR1 (or over expression to remain viable). In some examples, biological sample is screened for ROR1 over expression, for example over expression of ROR1 in leukemic cells in the sample, such as over expression of ROR1 in B-cells in the sample (e.g. B-cells that are arrested in the intermediate/mature stage of differentiation). Thus, in some embodiments, the disclosed methods include obtaining a biological sample from the subject, for example a sample of peripheral blood cells, such as a sample of white blood cells, for example as sample that includes B-cells.

In some examples, the amount of ROR1 expressed in the biological sample (for example a sample of peripheral blood cells, such as B-cells) is detected and compared to a control, such as a control indicative of a similar sample obtained from a subject who does not have a ROR1-dependent leukemia, such as a B-ALL, for example a t(1;19) B-ALL, a t(17;19) ALL or a t(8;14) Burkitt's ALL, or a reference value indicative of basal expression of ROR1 in the absence of ROR1-dependent leukemia, such as a B-ALL. If there is an increase in the amount of ROR1 expressed in the biological sample relative to the control (such as an amount of ROR1 expressed in a normal biological sample, for example a reference value or range of values representing the expected ROR1 expressed levels in a normal peripheral blood sample, such as a sample containing B-cells), the subject is diagnosed as having a ROR1-dependent leukemia, for example, an increase of at least 10%, at least 20%, at least 30%, at least 50%, at least 75%, at least 80%, at least 90%, at least 100%, at least 200% or even at least 500%, relative to the control, indicates that the subject (such as a human subject) has a ROR1-dependent leukemia.

Conversely, a reduction or maintenance in the amount of ROR1 expressed in the biological sample (for example a sample of peripheral blood cells, such as B-cells) relative to the control (such as an amount of ROR1 expressed in a normal biological sample, for example a reference value or range of values representing the expected ROR1 expressed levels in a normal peripheral blood, such as B-cells) indicates that the subject does not have a ROR1-dependent leukemia.

In some examples, cells obtained from the subject are contacted with an agent that inhibits ROR1 signaling activity. In some examples, the cells obtained from the subject are contacted with one or more inhibitory RNAs, such as siRNAs, that specifically inhibit expression of ROR1. In some examples, the cells obtained from the subject are contacted with one or more small molecule inhibitors of ROR1 signaling activity. In some examples, the cells obtained from the subject are contacted with an inhibitory antibody that specifically binds the extracellular domain of ROR1. After the cells are contacted with the molecule that inhibits the signaling activity of ROR1, a ROR1-regulated signaling activity is detected, such as one or more of cell proliferation, viability, or phosphorylation of ROR1 substrates or downstream targets in the signal transduction pathway in which ROR1 is a member. An alteration in the ROR1-regulated signaling activity in the cells obtained from the subject as compared to a control identifies the subject as one that would benefit from treatment with an agent that regulates ROR1 signaling activity, for example diagnoses the subject as having a ROR1-dependent leukemia, such as a ROR1-dependent B-ALL. In some examples, the alteration is a reduction in ROR1 signaling activity. In some examples, the alteration is a reduction in one or more of cell proliferation, cell viability or phosphorylation.

In some embodiments, the ROR1-regulated signaling activity is determined between about 1 hour and about 240 hours or longer after the cells are contacted with a molecule that inhibits the signaling activity of ROR1. For example, the ROR1-regulated signaling activity of the cells can be determined at about 1 hour, about 2 hours, about 3 hours, about 4 hours, about 5 hours, about 6 hours, about 12 hours, about 18 hours, about 24 hours, about 48 hours, about 72 hours, about 96 hours, about 120 hours, about 144 hours, about 168 hours, about 192 hours, about 216 hours, and about 240 hours, for example between about 1 hour and 12 hours, between about 5 hours and about 18 hours, between about 12 hours and about 72 hours, between about 48 hours and 168 hours after the cells are contacted with a molecule that inhibits the signaling activity of ROR1. Methods of detecting phosphorylation of proteins, such as phosphorylation of substrates of ROR1, are well known in the art and exemplary methods are given in Section H.

Exemplary methods of detecting cellular proliferation and/or viability, and conversely decreases in cellular proliferation and/or viability are given in Section G.

Examples of controls that can be used with the disclosed methods include statistical controls or cellular controls, such as white blood cells or bone marrow cells, obtained from the subject diagnosed with the leukemia, such as B-ALL that are not contacted with an agent that inhibits the signaling activity of ROR1. Alternatively, the controls are samples obtained from a second subject, such as subject that does not have a leukemia, such as a B-ALL. Examples of statistical controls are values that are indicative of basal proliferation or cell viability or phosphorylation. In some examples a control is a positive control, for example a cell-line that is dependent on ROR1, such as a RCH-ACV or a Kasumi-2 cell. In some examples, a control is a negative control, for example a cell-line that is not dependent on ROR1, such as a REH cell.

In some embodiments, the difference between the proliferation, viability of the cells and/or or phosphorylation of downstream targets in cells, contacted with an inhibitor of ROR1 signaling activity relative to a control is a decrease of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%, for example between about 10% and about 60%, between about 30% and about 90%, between about 60% and about 200%, between about 150% and about 400%, or between about 300% and about 500% reduced. In certain embodiments, for example where the signaling activity of ROR1 suppresses the phosphorylation of a downstream target in the biological signal transduction pathway that includes ROR1, the difference between phosphorylation of downstream targets in cells contacted with an inhibitor of ROR1 signaling activity relative to a control is an increase of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%, for example between about 10% and about 60%, between about 30% and about 90%, between about 60% and about 200%, between about 150% and about 400%, or between about 300% and about 500% reduced. In some embodiments, the difference relative to a control is a statistically significant difference.

In some embodiments, a therapeutic agent is selected that inhibits ROR1 signaling activity and the selected therapeutic agent is administered to the subject from whom the cells were obtained. In some examples, the selected therapeutic agent is a small molecule or antibody that inhibits the signaling activity of the ROR1. In some embodiments, a therapeutic agent that inhibits one or both of BTK and LYN kinase (such as dasatinib) is selected and the selected therapeutic agent is administered to the subject from whom the cells were obtained. Methods of treatment are disclosed in Section C.

Also disclosed are methods for monitoring a subject's response, such as a human subject's response, to a treatment for B-ALL. In such methods, a first biological sample (for example a sample of peripheral blood cells, such as B-cells,) is obtained at a first time point and a second biological sample (for example a sample of peripheral blood cells, such as B-cells) is obtained at second later time point from a subject being treated for B-ALL. The amount of ROR1 expressed in the first biological sample and in the second biological sample is detected and compared.

A decrease in the amount of ROR1 expressed in the second biological sample relative to the amount of ROR1 expressed in the first biological sample indicates that the subject is responding to the treatment for ROR1-dependent leukemia. For example, a reduction of at least 10%, at least 20%, at least 30%, at least 50%, at least 75%, at least 80%, at least 90%, at least 95%, at least 98% or even at least 99%, indicates that the subject is responding to treatment. Conversely an increase in or maintenance of the amount of ROR1 expressed in the second biological sample relative to the amount of ROR1 expressed in the first biological sample indicates that the subject is not responding to the treatment for ROR1-dependent leukemia. For example, an increase of at least 10%, at least 20%, at least 30%, at least 50%, at least 75%, at least 80%, at least 90%, at least 100%, at least 200% or even at least 500%, indicates that the subject is not responding to treatment.

### C. Methods of Treatment and Pharmaceutical Compositions

Methods are disclosed herein for treating a subject with a ROR1-dependent leukemia with an inhibitor of ROR1 signaling activity. The subject may be selected for example using the methods described in Section B. In some embodiments, a subject is selected that is suspected of having or having a t(1;19) B-ALL, a t(17;19) ALL or a t(8;14) Burkitt's ALL. Typical subjects intended for treatment with an inhibitor of ROR1 signaling activity include humans, as well as non-human primates and other animals, such as mice.

After selection, the subject is administered a therapeutically effective amount of an inhibitor of ROR1, BTK and/or LYN signaling activity, thereby treating the ROR1-dependent leukemia. Examples of inhibitors include inhibitory RNAs, small molecules (such as small molecule kinase inhibitors) and inhibitory antibodies. In some examples, the inhibitor of ROR1 signaling activity is provided as a pharmaceutical composition or compositions (see below). The administration of the inhibitor of ROR1 signaling activity can be for either a prophylactic or therapeutic purpose. When provided prophylactically, the inhibitor of ROR1, BTK and/or LYN signaling activity is provided in advance of any symptom. The prophylactic administration of the compounds serves to prevent or ameliorate any subsequent disease process. When provided therapeutically, the compounds are provided at (or shortly after) the onset of a disease or symptom of disease.

For prophylactic and therapeutic purposes, the inhibitor of ROR1, BTK and/or LYN signaling activity can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition.

Determination of effective dosages is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the inhibitor of ROR1, BTK and/or LYN signaling activity (for example, amounts that are effective to alleviate one or more symptoms of a targeted disease or condition). In alternative embodiments, an effective amount or effective dose of the inhibitor of ROR1, BTK and/or LYN signaling activity may simply inhibit or enhance one or more selected biological activities correlated with a disease or condition.

The actual dosage of the inhibitor of ROR1, BTK and/or LYN signaling activity will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the inhibitor of ROR1, BTK and/or LYN signaling activity for eliciting the desired signaling activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects. The specific dose level and frequency of dosage for any particular subject may be varied and will depend upon a variety of factors, including the activity of the specific compound, the extent of existing disease activity, the age, body weight, general health, sex, diet, mode and time of administration, rate of excretion, drug combination, and severity of the condition of the host undergoing therapy.

Compositions, such as therapeutic or pharmaceutical compositions, are provided that include an inhibitor of ROR1 BTK and/or LYN signaling activity. It is desirable to prepare the inhibitor of ROR1 signaling activity as a pharmaceutical composition appropriate for the intended application, for example to inhibit or treat a cellular proliferative disorder. Accordingly, methods for making a medicament or pharmaceutical composition containing an inhibitor of ROR1, BTK and/or LYN signaling activity are included herein. Inhibitors of ROR1, BTK and/or LYN signaling activity can be prepared for administration alone or with other active ingredients, such as other chemotherapeutics.

When an inhibitor of ROR1 signaling activity is administered in conjunction with an inhibitor of BTK and/or LYN signaling activity, the administration can be concurrent or sequential. Sequential administration can be separated by any amount of time, so long as the desired affect is achieved. Multiple administrations of the compositions described herein are also contemplated.

Pharmaceutical compositions including an inhibitor of ROR1, BTK and/or LYN signaling activity can be administered to subjects by a variety of routes. These include oral, nasal (such as intranasal), ocular, buccal, enteral, intravitral, or other mucosal (such as rectal or vaginal) or topical administration. Alternatively, administration will be by orthotopic, intradermal subcutaneous, intramuscular, parentral intraperitoneal, or intravenous injection routes. Such pharmaceutical compositions are usually administered as pharmaceutically acceptable compositions that include physiologically acceptable carriers, buffers or other excipients.

Typically, preparation of a pharmaceutical composition (for example, for use as a medicament or in the manufacture of a medicament) entails preparing a pharmaceutical composition that is essentially free of pyrogens, as well as any other impurities that could be harmful to humans or animals. An inhibitor of ROR1, BTK and/or LYN signaling activity may be included in pharmaceutical compositions (including therapeutic and prophylactic formulations), which are typically combined together with one or more pharmaceutically acceptable vehicles or carriers and, optionally, other therapeutic ingredients.

To formulate the pharmaceutical compositions, an inhibitor can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80), solubility enhancing agents (for example, cyclodextrins and derivatives thereof), stabilizers (for example, serum albumin), and reducing agents (for example, glutathione) can be included. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

An inhibitor of ROR1, BTK and/or LYN signaling activity can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, and microspheres.

An inhibitor of ROR1, BTK and/or LYN signaling activity can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

An inhibitor of ROR1, BTK and/or LYN signaling activity can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the inhibitor of ROR1, BTK and/or LYN signaling activity can be also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

For prophylactic and therapeutic purposes, the pharmaceutical compositions can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein.

Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of an inhibitor.

The appropriate dose will vary depending on the characteristics of the subject, for example, whether the subject is a human or non-human, the age, weight, and other health considerations pertaining to the condition or status of the subject, the mode, route of administration, and number of doses, time and route of administration, other drugs or treatments being administered concurrently, as well as the specific pharmacology of the therapeutic compositions for eliciting the desired activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects. A non-limiting range for a therapeutically effective amount of inhibitor of ROR1, BTK and/or LYN signaling activity within the methods and formulations of the disclosure is about 0.0001 μg/kg body weight to about 10 mg/kg body weight per dose, such as about 0.0001 μg/kg body weight to about 0.001 μg/kg body weight per dose, about 0.001 μg/kg body weight to about 0.01 μg/kg body weight per dose, about 0.01 μg/kg body weight to about 0.1 μg/kg body weight per dose, about 0.1 μg/kg body weight to about 10 μg/kg body weight per dose, about 1 μg/kg body weight to about 100 μg/kg body weight per dose, about 100 μg/kg body weight to about 500 μg/kg body weight per dose, about 500 μg/kg body weight per dose to about 1000 μg/kg body weight per dose, or about 1.0 mg/kg body weight per dose to about 10 mg/kg body weight per dose.

Antibodies, for example inhibitory antibodies that specifically bind the extracellular domain of ROR1, may be provided in lyophilized form and rehydrated with sterile water before administration, although they are also provided in sterile solutions of known concentration. In some examples, the antibody solution is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXAN® in 1997. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Therapeutic compositions that include an inhibitor of ROR1, BTK and/or LYN signaling activity can be delivered by way of a pump (see Langer, supra; Sefton, CRC Crit. Ref. Biomed. Eng. 14:201, 1987; Buchwald et al., Surgery 88:507, 1980; Saudek et al., N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, a pump is implanted (for example see U.S. Pat. Nos. 6,436,091; 5,939,380; and 5,993,414). Implantable drug infusion devices are used to provide patients with a constant and long-term dosage or infusion of a therapeutic agent. Such device can be categorized as either active or passive.

Active drug or programmable infusion devices feature a pump or a metering system to deliver the agent into the patient's system. An example of such an active infusion device currently available is the Medtronic SYNCHROMED™ programmable pump. Passive infusion devices, in contrast, do not feature a pump, but rather rely upon a pressurized drug reservoir to deliver the agent of interest. An example of such a device includes the Medtronic ISOMED™.

In particular examples, therapeutic compositions are administered by sustained-release systems. Suitable examples of sustained-release systems include suitable polymeric materials (such as, semi-permeable polymer matrices in the form of shaped articles, for example films, or microcapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res. 15:167-277, 1981; Langer, Chem. Tech. 12:98-105, 1982, ethylene vinyl acetate (Langer et al., Id.) or poly-D-(−)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, poloxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (for example, U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

### D. Detection of ROR1 Protein Molecules

In some embodiments of the disclosed methods, determining the amount of ROR1 expressed in a biological sample includes determining the amount of ROR1 protein, such as a ROR1 protein with an amino acid sequence at least about 80% identical, such as at least about 85% identical, at least about 90% identical, at least about 95% identical, at least about 98% identical, or even about 100% identical to SEQ ID NO. 1 or a fragment thereof, in the biological sample.

ROR1 protein can be detected and the amount of ROR1 protein present in the biological sample can be detected through novel epitopes recognized by polyclonal and/or monoclonal antibodies used in ELISA assays, immunoblot assays, flow cytometric assays, immunohistochemical assays, radioimmuno assays, Western blot assays, an immunofluorescent assays, chemiluminescent assays and other polypeptide detection strategies (Wong et al., Cancer Res., 46: 6029-6033, 1986; Luwor et al., Cancer Res., 61: 5355-5361, 2001; Mishima et al., Cancer Res., 61: 5349-5354, 2001; Ijaz et al., J. Med. Virol., 63: 210-216, 2001). Generally these methods utilize antibodies, such as monoclonal or polyclonal antibodies.

Generally, immunoassays for ROR1 typically include incubating a biological sample in the presence of antibody, and detecting the bound antibody by any of a number of techniques well known in the art. The biological sample can be brought in contact with and immobilized onto a solid phase support or carrier such as nitrocellulose, or other solid support which is capable of immobilizing cells, cell particles or soluble proteins. The support may then be washed with suitable buffers followed by treatment with the antibody that binds ROR1. The solid phase support can then be washed with the buffer a second time to remove unbound antibody. If the antibody is directly labeled, the amount of bound label on solid support can then be detected by conventional means. If the antibody is unlabeled, a labeled second antibody, which detects that antibody that specifically binds ROR1 can be used.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros and magnetite. The nature of the carrier can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet or test strip.

In one embodiment, an enzyme linked immunosorbent assay (ELISA) is utilized to detect the protein (Voller, “The Enzyme Linked Immunosorbent Assay (ELISA),” Diagnostic Horizons 2:1-7, 1978, Microbiological Associates Quarterly Publication, Walkersville, Md.; Voller et al., J. Clin. Pathol. 31:507-520, 1978; Butler, Meth. Enzymol. 73:482-523, 1981; Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). In this method, an enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection can also be accomplished by visual comparison of the extent of enzymatic reaction of a substrate in comparison with similarly prepared standards.

Detection can also be accomplished using any of a variety of other immunoassays. For example, by radioactively labeling the antibodies or antibody fragments, it is possible to detect fingerprint gene wild-type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). In another example, a sensitive and specific tandem immunoradiometric assay may be used (see Shen and Tai, J. Biol. Chem., 261:25, 11585-11591, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In some embodiments, the amount of ROR1 protein present in the biological sample and thus the amount of ROR1 expressed is detected using a ROR1 protein specific binding agent, such as an antibody or ligand for ROR1, which can be detectably labeled. In some embodiments, the specific binding agent is an antibody, such as a polyclonal or monoclonal antibody, that specifically binds ROR1 protein. Thus in certain embodiments, determining the amount of ROR1 expressed in a biological sample includes contacting a biological sample from the subject with a ROR1 protein specific binding agent (such as an antibody that specifically binds ROR1 protein), detecting whether the binding agent is bound by the sample, and thereby measuring the amount of ROR1 protein present in the sample. In certain embodiments, the ROR1 protein specific binding agent is an antibody or an antibody fragment that specifically binds ROR1 protein. In one embodiment, the specific binding agent is a monoclonal or polyclonal antibody that specifically binds the ROR1 protein.

An antibody that specifically binds a ROR1 protein typically binds with an affinity constant of at least 10⁷ M^{- 1}, such as at least 10⁸ M⁻¹ at least 5×10⁸ M⁻¹ or at least 10⁹ M⁻¹. All of these antibodies are of use in the methods disclosed herein.

The preparation of polyclonal antibodies is well known to those skilled in the art. See, for example, Green et al., “Production of Polyclonal Antisera,” in: Immunochemical Protocols pages 1-5, Manson, ed., Humana Press 1992; Coligan et al., “Production of Polyclonal Antisera in Rabbits, Rats, Mice and Hamsters,” in: Current Protocols in Immunology, section 2.4.1, 1992.

The preparation of monoclonal antibodies likewise is conventional. See, for example, Kohler & Milstein, Nature 256:495, 1975; Coligan et al., sections 2.5.1-2.6.7; and Harlow et al., in: Antibodies: a Laboratory Manual, page 726, Cold Spring Harbor Pub., 1988. Briefly, monoclonal antibodies can be obtained by injecting mice with a composition including an antigen or a cell of interest, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG),” in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993.

Antibodies include intact molecules as well as functional fragments thereof, such as Fab, F(ab′)₂, and Fv which are capable of binding the epitopic determinant. These antibody fragments retain some ability to selectively bind with their antigen. Methods of making these fragments are known in the art. (See for example, Harlow and Lane, Antibodies: A Laboratory Manual, Cold Spring Harbor Laboratory, New York, 1988).

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab−Ag]/[Ab][Ag] where [Ab] is the concentration at equilibrium of the antibody, [Ag] is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

The antibodies used in the methods disclosed herein can be labeled. The enzymes that can be conjugated to the antibodies include, but are not limited to, alkaline phosphatase, peroxidase, urease and B-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P., Molecular Probes Handbook of Fluorescent Probes and Research Chemicals (1992-1994). The metal compounds that can be conjugated to the antibodies include, but are not limited to, ferritin, colloidal gold, and particularly colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc), ¹²⁵I and amino acids including any radionucleotides, including but not limited to, ¹⁴C, ³H and ³⁵S.

Any method known to those of skill in the art can be used to detect and quantify ROR1 protein. Thus, in additional embodiments, a spectrometric method is utilized. Spectrometric methods include mass spectrometry, nuclear magnetic resonance spectrometry, and combinations thereof. In one example, mass spectrometry is used to detect the presence of ROR1 protein in a biological sample, such as a blood sample, a serum sample, or a plasma sample (see for example, Stemmann, et al., Cell December 14; 107(6):715-26, 2001; Zhukov et al., “From Isolation to Identification: Using Surface Plasmon Resonance-Mass Spectrometry in Proteomics, PharmaGenomics, March/April 2002, available on the PharmaGenomics website on the internet).

ROR1 protein also can be detected by mass spectrometry assays for example coupled to immunoaffinity assays, the use of matrix-assisted laser desorption/ionization time-of-flight (MALDI-TOF) mass mapping and liquid chromatography/quadrupole time-of-flight electrospray ionization tandem mass spectrometry (LC/Q-TOF-ESI-MS/MS) sequence tag of proteins separated by two-dimensional polyacrylamide gel electrophoresis (2D-PAGE) (Kiernan et al., Anal. Biochem., 301: 49-56, 2002; Poutanen et al., Mass Spectrom., 15: 1685-1692, 2001).

The presence of a ROR1 protein can be detected with multiple specific binding agents, such as one, two, three, or more specific binding agents. Thus, the methods can utilize more than one antibody. In some embodiments, one of the antibodies is attached to a solid support, such as a multiwell plate (such as, a microtiter plate), bead, membrane or the like. In practice, microtiter plates may conveniently be utilized as the solid phase. The surfaces may be prepared in advance, stored, and shipped to another location(s). However, antibody reactions also can be conducted in a liquid phase.

### E. Detection of ROR1 Nucleic Acid Molecules

In some embodiments of the disclosed methods, determining the amount of ROR1 expressed in a biological sample includes determining the amount of ROR1 nucleic acids, such as ROR1 mRNA, in the biological sample. For example a ROR1 nucleic acid with an nucleic acid sequence at least about 80% identical, such as at least about 85% identical, at least about 90% identical, at least about 95% identical, at least about 98% identical, or even about 100% identical to SEQ ID NO: 2 or a fragment thereof, in the biological sample.

Methods of determining the amount of nucleic acids, such mRNA encoding ROR1 based on hybridization analysis and/or sequencing are known in the art. The most commonly used methods known in the art for the quantification of mRNA expression in a sample include northern blotting and in situ hybridization (Parker & Barnes, Methods in Molecular Biology 106:247 283, 1999); RNAse protection assays (Hod, Biotechniques 13:852 854 (1992)); and PCR-based methods, such as reverse transcription polymerase chain reaction (RT-PCR) (Weis et al., Trends in Genetics 8:263 264, 1992). Representative methods for sequencing-based gene expression analysis include Serial Analysis of Gene Expression (SAGE), and gene expression analysis by massively parallel signature sequencing (MPSS). In some embodiments, determining the amount of ROR1 expressed in a biological sample includes determining the amount of ROR1 mRNA in the biological sample.

Methods for quantitating mRNA are well known in the art. In one example, the method utilizes reverse transcriptase polymerase chain reaction (RT-PCR). Generally, the first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP® RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using commercially available equipment, such as, for example, ABI PRISM 7700® Sequence Detection System™ (Perkin-Elmer-Applied Biosystems, Foster City, Calif., USA), or Lightcycler (Roche Molecular Biochemicals, Mannheim, Germany). In one embodiment, the 5′ nuclease procedure is run on a real-time quantitative PCR device such as the ABI PRISM 7700® Sequence Detection System. The system includes a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

In some examples, 5′-nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR can be performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH), beta-actin, and 18S ribosomal RNA.

Generally, with regard to nucleic acids, any method can be utilized provided it can detect the expression of target gene mRNA (ROR1) as compared to a control. One of skill in the art can readily identify an appropriate control, such as a sample from a subject known not to have a disorder (a negative control), a sample from a subject known to have a disorder (a positive control), or a known amount of nucleic acid encoding ROR1 (a standard or a normal level found in a healthy subject). Statistically normal levels can be determined for example, from a subject with known not to have B-ALL.

The methods described herein may be performed, for example, by utilizing pre-packaged diagnostic kits comprising at least one specific nucleic acid probe, which may be conveniently used, such as in clinical setting. In one embodiment, this assay is performed in a medical laboratory on a sample of peripheral blood, cells isolated from the peripheral blood, serum or plasma.

The diagnostic procedures can be performed “in situ” directly upon blood smears (fixed and/or frozen), or on tissue biopsies, such that no nucleic acid purification is necessary. DNA or RNA from a sample can be isolated using procedures which are well known to those in the art.

Nucleic acid reagents that are specific to the nucleic acid of interest, namely the nucleic acid encoding ROR1, can be readily generated given the sequences of the gene (such as the nucleic acid sequence set forth as SEQ ID NO: 2) for use as probes and/or primers for such in situ procedures (see, for example, Nuovo, G. J., 1992, PCR in situ hybridization: protocols and applications, Raven Press, NY).

In one embodiment, a nucleic acid sample is utilized, such as the total mRNA isolated from a biological sample. The biological sample can be from any biological tissue or fluid from the subject of interest, such as a subject who is suspected of having cardiovascular disease. Such samples include, but are not limited to, blood, blood cells (such as white blood cells).

Nucleic acids (such as mRNA) can be isolated from the sample according to any of a number of methods well known to those of skill in the art. Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology Hybridization With Nucleic Acid Probes, Part I. Theory and Nucleic Acid Preparation, P. Tijssen, ed. Elsevier, N.Y. (1993). In one example, the total nucleic acid is isolated from a given sample using, for example, an acid guanidinium-phenol-chloroform extraction method, and polyA+ mRNA is isolated by oligo dT column chromatography or by using (dT)n magnetic beads (see, for example, Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, N.Y. (1987)). In another example, oligo-dT magnetic beads may be used to purify mRNA (Dynal Biotech Inc., Brown Deer, Wis.).

The nucleic acid sample can be amplified prior to hybridization. If a quantitative result is desired, a method is utilized that maintains or controls for the relative frequencies of the amplified nucleic acids. Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. The array can then include probes specific to the internal standard for quantification of the amplified nucleic acid.

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (such as fluorescein-labeled UTP and/or CTP) incorporates a label into the transcribed nucleic acids.

Alternatively, a label may be added directly to the original nucleic acid sample (such as mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. These steps have been standardized for commercially available array systems.

Methods for evaluating the hybridization results vary with the nature of the specific probe nucleic acids used as well as the controls provided. In one embodiment, simple quantification of the fluorescence intensity for each probe is determined. This is accomplished simply by measuring probe signal strength at each location (representing a different probe) on the array (for example, where the label is a fluorescent label, detection of the amount of florescence (intensity) produced by a fixed excitation illumination at each location on the array). Comparison of the absolute intensities of an array hybridized to nucleic acids from a “test” sample (such as from a subject treated with a therapeutic protocol) with intensities produced by a “control” sample (such as from the same subject prior to treatment with the therapeutic protocol) provides a measure of the relative expression of the nucleic acids that hybridize to each of the probes.

Changes in expression detected by these methods for instance can be different for different therapies, and may include increases or decreases in the level (amount) or functional activity of such nucleic acids, their expression or translation into protein, or in their localization or stability. An increase or a decrease can be, for example, about a 1-fold, 2-fold, 3-fold, 4-fold, 5-fold, change (increase or decrease) in the expression of a particular nucleic acid, such as a nucleic acid encoding ROR1.

Alterations, including increases or decreases in the expression of nucleic acid molecules can be detected using, for instance, in vitro nucleic acid amplification and/or nucleic acid hybridization. The results of such detection methods can be quantified, for instance by determining the amount of hybridization or the amount of amplification.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

### F. Screening Methods

Methods are provided herein for identifying an agent of use in treating a subject with a ROR1-dependent leukemia, such as a ROR1-dependent ALL, for example a B-ALL. The methods include contacting an isolated test cell that is dependent on ROR1 signaling activity with a test agent and detecting a ROR1-regulated signaling activity. The amount of the ROR1-regulated signaling activity in the test cell is compared to a control. An alteration in the ROR1-regulated signaling activity in the test cell relative to the control indicates that the agent is useful for the treatment of a subject with ROR1-dependent leukemia or with a predisposition for developing a ROR1-dependent leukemia. The isolated cell can be any cell of interest, including human and non-human cells. For example, the cells can be mouse, rat, monkey, or human cells.

A variety of controls can be used in these methods. In some embodiments, the control is a standard value. In other embodiments, the control is ROR1-regulated signaling activity in a cell not contacted with the agent or contacted with an agent known not to affect ROR1 signaling activity. In some examples, a control is a cell that is not ROR1-dependent.

In one embodiment, for a high throughput format, cells can be introduced into wells of a multi-well plate or of a glass slide or microchip, and can be contacted with the test agent. Generally, the cells are organized in an array, particularly an addressable array, such that robotics conveniently can be used for manipulating the cells and solutions and for monitoring the cells, particularly with respect to the function being examined, such as ROR1 signaling activity. An advantage of using a high throughput format is that a number of test agents can be examined in parallel, and, if desired, control reactions also can be run under identical conditions as the test conditions. As such, the methods disclosed herein provide a means to screen one, a few, or a large number of test agents in order to identify an agent that can alter a function of cells, for example, an agent that inhibits ROR1 signaling activity.

Any suitable compound or composition can be used as a test agent, such as organic or inorganic chemicals, including aromatics, fatty acids, and carbohydrates; peptides, including monoclonal antibodies, polyclonal antibodies, and other specific binding agents; phosphopeptides; or nucleic acid molecules. In a particular example, the test agent includes a random peptide library (for example see Lam et al., Nature 354:82-4, 1991), random or partially degenerate, directed phosphopeptide libraries (for example see Songyang et al., Cell 72:767-78, 1993). A test agent can also include a complex mixture or “cocktail” of molecules.

Test agents can be pharmacologic agents already known in the art or can be compounds previously unknown to have any pharmacological activity. The compounds can be naturally occurring or designed in a laboratory. They can be isolated from microorganisms, animals, or plants, and can be produced recombinantly, or synthesized by chemical methods known in the art. If desired, test substances can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds (for example see Lam, Anticancer Drug Des. 12:145, 1997).

Methods for the synthesis of molecular libraries are well known in the art (see, for example, DeWitt et al., Proc. Natl. Acad. Sci. U.S.A. 90:6909, 1993; Erb et al. Proc. Natl. Acad. Sci. U.S.A. 91:11422, 1994; Zuckermann et al., J. Med. Chem. 37:2678, 1994; Cho et al., Science 261:1303, 1993; Carell et al., Angew. Chem. Int. Ed. Engl. 33:2059, 1994; Carell et al., Angew. Chem. Int. Ed. Engl. 33:2061; Gallop et al., J. Med. Chem. 37:1233, 1994). Libraries of compounds can be presented in solution (see, for example, Houghten, BioTechniques 13:412-21, 1992), or on beads (Lam, Nature 354, 82-4, 1991), chips (Fodor, Nature 364:555-6, 1993), bacteria or spores (Ladner, U.S. Pat. No. 5,223,409), plasmids (Cull et al., Proc. Natl. Acad. Sci. U.S.A. 89:1865-9, 1992), or phage (Scott & Smith, Science 249:386-90, 1990; Devlin, Science 249:404-6, 1990); Cwirla et al., Proc. Natl. Acad. Sci. 97:6378-82, 1990; Felici, J. Mol. Biol. 222:301-10, 1991; and Ladner, U.S. Pat. No. 5,223,409).

### G. Detection of Cellular Proliferation and Viability

The methods provided herein can involve determining if proliferation and/or viability of test cells and/or cells obtained from a subject (such as white blood cells and/or bone marrow cells) is inhibited. The cells are contacted with an agent that inhibits the signaling activity of ROR1, BTK and/or LYN, for example small molecule, antisense inhibitors or test agents. Following contact with an agent, the cells are assayed for proliferation and/or viability, for example by assaying one or more of growth, apoptosis and necrosis.

In one example, the cells are labeled with acridine orange and ethidium bromide and subjected to florescent microscopy following labeling. An increase in apoptosis or decrease in viability of test cells contacted with an agent relative to a control can be used to identify an agent of use in treating a ROR1-dependent leukemia, such as a ROR1 dependent B-ALL. Similarly, an increase in apoptosis or decrease in viability of cells obtained from a subject contacted with an agent the alters the signaling activity of ROR1 relative to a control can be used to identify a subject as one that would benefit from the administration of an inhibitor of ROR1 BTK and/or LYN signaling activity. Many methods for measuring cellular proliferation and/or viability are known to those of ordinary skill in the art.

For example, floating cells can be collected by trypsinization and washed three times in PBS. Aliquots of cells are then centrifuged. The pellet is resuspended in media and a dye mixture containing acridine orange and ethidium bromide prepared in PBS and mixed gently. The mixture then can be placed on a microscope slide and examined for morphological features of apoptosis.

Apoptosis also can be quantified by measuring an increase in DNA fragmentation in cells that have been treated with test compounds. Commercial photometric enzyme immunoassays (EIA) for the quantitative in vitro determination of cytoplasmic histone-associated-DNA-fragments (mono- and oligo-nucleosomes) are available (for example, Cell Death Detection ELISA, Boehringer Mannheim). The Boehringer Mannheim assay is based on a sandwich-enzyme-immunoassay principle, using mouse monoclonal antibodies directed against DNA and histones, respectively. This allows the specific determination of mono- and oligo-nucleosomes in the cytoplasmic fraction of cell lysates. According to the vendor, apoptosis is measured as follows. The sample (cell-lysate) is placed into a streptavidin-coated microtiter plate (“MTP”). Subsequently, a mixture of anti-histone-biotin and anti-DNA peroxidase conjugates is added and incubated for two hours. During the incubation period, the anti-histone antibody binds to the histone-component of the nucleosomes and simultaneously fixes the immunocomplex to the streptavidin-coated MTP via its biotinylation. Additionally, the anti-DNA peroxidase antibody reacts with the DNA component of the nucleosomes. After removal of unbound antibodies by a washing step, the amount of nucleosomes is quantified by the peroxidase retained in the immunocomplex. Peroxidase is determined photometrically with ABTS7 (2,2′-Azido-[3-ethylbenzthiazolin-sulfonate]) as substrate. An increase in apoptosis relative to a control can be at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%, for example between about 10% and about 60%, between about 30% and about 90%, between about 60% and about 200%, between about 150% and about 400%, or between about 300% and about 500%.

In another example, proliferation and/or cell viability is measured by incorporation of radioactive tritium into proliferating cells. Radioactive tritium is not incorporated into non-viable cells. Cells obtained from a subject, such as blood cells and/or bone marrow cells are cultured for a period of time (such as from at least about 2 hours to at least about 4 days, for example at least about 18 hours), with 0.25 μCi of [³H] thymidine, harvested onto glass filters, and radionucleotide incorporation is measured with a liquid scintillation counter. The amount of [³H] thymidine incorporated into the cells is proportional to the proliferation of the cells. A decrease in cell proliferation relative to a control can be at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%, for example between about 10% and about 60%, between about 30% and about 90%, between about 60% and about 200%, between about 150% and about 400%, or between about 300% and about 500%.

Another example of a method of measuring cell viability and/or proliferation is the incorporation of bromodeoxyuridine (BrdU), a thymidine analog, into newly synthesized DNA strands of actively proliferating cells. In contrast BrdU is not incorporated into non-viable cells. BrdU is detected immunochemically allowing the assessment of the population of cells, which are actively synthesizing DNA, for example using the CALBIOCHEM® BrdU Cell Proliferation Assay. According to the manufacturer, BrdU is added to cells and will be incorporated into the DNA of dividing cells. The cells are fixed and permeabilized and the DNA denatured to enable an anti-BrdU monoclonal antibody to bind the BrdU containing DNA. In some examples, an anti-BrdU monoclonal antibody is allowed to incubate with the cells for 1 hour, during which time it binds to any incorporated BrdU. Unbound antibody is washed away and horseradish peroxidase-conjugated goat anti-mouse is added, which binds to the detector antibody. The horseradish peroxidase catalyzes the conversion of the chromogenic substrate tetra-methylbenzidine (TMB) from a colorless solution to a blue solution (or yellow after the addition of stopping reagent), the intensity of which is proportional to the amount of incorporated BrdU in the cells. The colored reaction product is quantified using a spectrophotometer. A decrease in the amount of BrdU incorporated relative to a control can be at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%, for example between about 10% and about 60%, between about 30% and about 90%, between about 60% and about 200%, between about 150% and about 400%, or between about 300% and about 500%.

Another method of measuring cell viability is the ability of cells to exclude propidium iodide (PI). The integrity of the plasma membrane and hence the viability of cell can be assessed by determining the ability of cells to exclude PI from the interior of the cells. Typically, cells are collected by centrifugation, washed once with PBS, and resuspended in PBS containing 1 μg of PI/ml. The level of PI incorporation into cells can be quantified by flow cytometry, for example on a FACSCAN® flow cytometer GUAVA TECHNOLOGIES® flow cytometer. A decrease in the number of cells that can exclude relative to a control can be at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%, for example between about 10% and about 60%, between about 30% and about 90%, between about 60% and about 200%, between about 150% and about 400%, or between about 300% and about 500%.

In other examples, cell proliferation is measured with a 3-(4,5-dimethylthiazol-2-yl)-5-(3-carboxymethoxyphenyl)-2-(4-sulfophenyl)-2H-tetrazolium (MTS) cell proliferation assay. An MTS cell proliferation assay is a colorimetric method to identify the cytotoxic potential of a test item. After contacting a sample containing cells with MTS, the formation of a soluble formazan (which is a product MTS) is measured after a period of time. Methods for measuring formazan, such as spectroscopic methods, are well known in the art. The amount of soluble formazan is directly proportional to the number of live cells in the sample. Thus, if the amount of soluble formazan increases as function of time the cell are proliferating. Conversely, if the amount of soluble formazan decreases as function of time the cell are proliferating. The rate of cell proliferation can be calculated by determining the change in the amount of formazan a first time point to the amount of at a second later time point. A decrease in the amount of formazan relative to a control can be at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%, for example between about 10% and about 60%, between about 30% and about 90%, between about 60% and about 200%, between about 150% and about 400%, or between about 300% and about 500%.

### H. Method of Detecting Phosphorylation

The methods provided herein can involve determining if phosphorylation of downstream targets the biological signal transduction pathway containing ROR1 and/or BTK and LYN are altered by contacting the cells with an agent that inhibits the signaling activity of ROR1, for example small molecule, antisense inhibitors of ROR1, BTK and/or LYN signaling activity or test agents. Following contact with an agent, the cells are assayed for alterations in the phosphorylation of downstream targets in the biological signal transduction pathway.

The presence of alterations in phosphorylation can be determined using any method known to one of skill in the art. In several examples, the presence of phosphorylation is determined using an antibody, such as a monoclonal or polyclonal antibody, that specifically binds a phosphorylated protein or peptide in the ROR1, BTK and/or LYN biological signal transduction pathway when the protein or peptide phosphorylated. The presence of antibody:antigen complexes can be determined using methods known in the art. For example, the antibody can include a detectable label, such as a fluorophore, radiolabel, or enzyme, which permits detection of the antibody, for example using enzyme-linked immunosorbent assay (ELISA).

An ELISA is a biochemical technique that can be used mainly to detect the presence of an antibody or an antigen in a sample, for example an antibody that specifically binds a phosphorylated peptide or protein, such as a phosphorylated peptide or protein in the biological signal transduction pathway that includes ROR1, BTK and/or LYN. In some examples, the antibody can be linked to an enzyme, for example directly conjugated or through a secondary antibody, and a substance is added that the enzyme can convert to a detectable signal. Thus, in the case of fluorescence ELISA, when light of the appropriate wavelength is shone upon the sample, any antigen:antibody complexes will fluoresce so that the amount of antigen in the sample can be inferred through the magnitude of the fluorescence. The antigen is usually immobilized on a solid support (for example polystyrene microtiter plate) either non-specifically (for example via adsorption to the surface) or specifically (for example via capture by another antibody specific to the same antigen, in a “sandwich” ELISA). Between each step the plate is typically washed with a mild detergent solution, such as phospho-buffered saline with or without NP40 or TWEEN© to remove any proteins or antibodies that are not specifically bound. After the final wash step the plate is developed by adding an enzymatic substrate to produce a visible signal, which indicates the quantity of antigen in the sample.

In some examples, the method can include contacting the sample with a second antibody that specifically binds to the first antibody that specifically binds a phosphorylated peptide or protein, such as a phosphorylated peptide or protein in the biological signal transduction pathway that includes ROR1, BTK and/or LYN. In some examples, the second s antibody is detectably labeled, for example with a fluorophore (such as FTIC, PE, a fluorescent protein, and the like), an enzyme (such as HRP), a radiolabel, or a nanoparticle (such as a gold particle or a semiconductor nanocrystal, such as a quantum dot (QDOT®)).

Phosphorylated proteins can be detected for example using stains specific for phosphorylated proteins in gels. Phosphorylated proteins can be detected by measuring the transfer of a labeled phosphate (such as radioactive phosphorus (³²P)) from the γ phosphate of a nucleotide triphosphate, such as ATP to the peptide. By addition of γ phosphate labeled triphosphate, such as ATP, into sample, the presence of phosphorylated peptide can be determined. In some embodiments, a γ phosphate labeled triphosphate, such as ATP is added to the sample. Typically, the γ phosphate label is a radioisotope label, although any label that can be transferred via a kinase reaction is contemplated by this disclosure.

The amount of the phosphorylated protein can be compared to a control. In several embodiments, the control is a known value indicative of basal phosphorylation of the protein, for example a value found in the absence of ROR1 signaling activity.

In some embodiments, the difference in the amount of phosphorylated protein relative to a control is a statistically significant difference. In some embodiments, the difference in the amount of phosphorylated peptide relative to a control is at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

### I. Computer-Based Implementation of Certain Embodiments

As used herein, “a computer-based system” refers to the hardware means, software means, and data storage means used to analyze information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

The analytic methods described herein can be implemented by use of computer systems. For example, any of the comparison steps described above may be performed by means of software components loaded into a computer or other information appliance or digital device. When so enabled, the computer, appliance or device may then perform the above-described steps to assist the analysis of values associated with a one or more genes or protein expression or protein activity (for example a value that correlates with the measured activity of a particular protein, such as ROR1 signaling activity), or for comparing such associated values. The above features are embodied in one or more computer programs may be performed by one or more computers running such programs.

The minimum hardware of the computer-based systems of the present invention comprises a central processing unit (CPU), input means, output means, and data storage means. For example, an exemplary computer system suitable for implementation of the analytic methods of this invention includes internal components and is linked to external components. The internal components of this computer system include a processor element interconnected with main memory. The external components include mass storage. This mass storage can be one or more hard disks (which are typically packaged together with the processor and memory). Such hard disks are preferably of 1 GB or greater storage capacity. Other external components include user interface device, which can be a monitor, together with inputting device, which can be a “mouse”, or other graphic input devices, and/or a keyboard. A printing device can also be attached to the computer. Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on mass storage. The software components include an operating system, which is responsible for managing the computer system and its network interconnections. This operating system can be, for example, of the Microsoft Windows' family, such as Windows 7, or earlier or later versions. The software components also include common languages and functions conveniently present on this system to assist programs implementing the methods specific to this invention. Many high or low level computer languages can be used to program the analytic methods of this invention. Instructions can be interpreted during run-time or compiled. Preferred languages include C/C++, FORTRAN and JAVA. Most preferably, the methods of this invention are programmed in mathematical software packages that allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), and S-Plus from Math Soft (Cambridge, Mass.).

Alternative computer systems and software for implementing the analytic methods of this invention will be apparent to one of skill in the art.

### EXAMPLES

### Example 1

### Identification of ROR1 as a Target and Marker for Acute Lymphocytic Leukemia

To identify functionally important oncogenes and therapeutic agents for ALL, functional profiling of primary cells from ALL patients was performed with panels of siRNAs and small-molecule kinase inhibitors. Follow-up analyses were also performed on patient-derived cell lines and primary xenograft cells from ALL patients.

Universal overexpression and functional dependence on ROR1 was identified. A sensitivity to the kinase inhibitor dasatinib was also identified in t(1;19) ALL patient-derived cell lines, primary xenograft cells, and primary patient samples. Follow-up tests identified ROR1 overexpression and dasatinib sensitivity in other subsets of B-cell malignancies that are also arrested at intermediate or mature stages of B-cell development, such as (t(17;19)/ALL and t(8;14)/Burkitt's). Further examination revealed that dasatinib treatment impairs signaling from the pre-B-cell receptor (pre-BCR) via inhibition of BTK and LYN kinases and induces further upregulation of ROR1 expression. Silencing of ROR1 followed by dasatinib treatment accentuated dasatinib killing of t(1;19) cells.

### Methods

Patient Samples:

Bone marrow mononuclear cells were separated on a Ficoll gradient. Cells were cultured in RPMI-1640 medium supplemented with 20% FBS (Atlanta Biologicals, Lawrenceville, Ga.), L-glutamine, insulin/transferrin/sodium selenite, penicillin/streptomycin, fungizone (Invitrogen, Carlsbad, Calif.), and 10⁻⁴M 2-mercaptoethanol (Sigma).

Primary Leukemia Cell Xenograft:

2×10⁶ cells from a bone marrow biopsy of a patient with t(1;19)-positive ALL were inoculated via tail vein injection into sublethally irradiated (250 cGy) NOD/SCID mice. When the mice became terminally ill because of overt leukemia, they were sacrificed and leukemia cells were harvested from bone marrow and spleen. Leukemic infiltration was confirmed by flow cytometry and cells were suspended in culture media (RPMI-1640 medium supplemented with 20% FBS, L-glutamine, insulin/transferrin/sodium selenite, penicillin/streptomycin, fungizone, and β-mercaptoethanol).

Cell Culture:

RCH-ACV, Kasumi-2, MHH-cALL2 and REH cells were obtained from DSMZ and cultured in RPMI supplemented with 10% FBS (or 20% for MHH-cALL2), penicillin/streptomycin, L-glutamine, and fungizone.

Gene Expression Microarray:

A meta-analysis was performed for gene expression microarray analyses of pediatric ALL patient samples and normal B-cell progenitor populations. Datasets were processed and normalized using the RMA algorithm and normalization was validated based on even expression levels for a set of 7 reference genes (HPRT, COX6B, GUSB, GAPDH, PGK, ACTB and B2M) among all tissue samples studied. Gene expression values for the ROR1 probesets (211057_at and 205805_s_at) and the IGHM probeset (212827_at) were studied.

siRNA and RAPID Assay:

The RAPID assay and other siRNA experiments were performed described in Tyner et al., Proc Natl Acad Sci USA. 2009 May 26; 106(21):8695-8700, which is incorporated herein by reference in its entirety. All siRNAs were from Thermo Fisher Scientific Dharmacon RNAi Technologies. RT-PCR. Total RNA (Qiagen®, RNeasy) from each sample was used to synthesize cDNA (Invitrogen® SuperScript® III) with random hexamer primers.

Immunoblotting and Flow Cytometry:

All cells were lysed in sample buffer (75 mM Tris pH 6.8, 3% SDS, 15% glycerol, 8% β-mercaptoethanol, 0.1% bromophenol blue) and separated by SDS-PAGE. Proteins were transferred to PVDF membranes (Millipore, Billerica, Mass.) and subjected to immunoblot analysis with antibodies specific for ROR1 (R&D Systems, Minneapolis, Minn.), E2A-PBX1 (BD Biosciences, San Jose, Calif.), BCL6, phospho-AKT (Cell Signaling), or β-actin (Millipore). For flow cytometry, cells were immunostained with antibody specific for ROR1 (R&D Systems) or Goat IgG (Jackson ImmunoResearch), washed 3 times with PBS wash buffer containing 2% FBS, then stained with Donkey Anti-goat IgG-Phycoerythrin (R&D Systems). Samples were analyzed on a BD FACSAria®.

Cell Viability Experiments:

For proliferation and viability assays, cells were electroporated with 1-2 μM siRNA at 250 V, 0.2 ms, 2 pulses and incubated for 96 hours or cells were incubated in the presence of dose gradients of dasatinib (LC Labs, Woburn, Mass.) for 72 hours (density per well of 5,000 for cell lines and 50,000 for primary cells) at which point a CellTiter® 96 AQᵤₑₒᵤₛ One solution cell proliferation assay was performed (Promega, Madison, Wis.). Absorbance values were normalized to control wells containing cells electroporated with non-specific siRNA or in the absence of drug. For apoptosis studies, cells were stained with Guava Nexin Reagent (Millipore) and analyzed on a Guava EasyCyte Plus Flow Cytometry System (Millipore).

Statistical Analyses:

For RAPID screens, the mean and standard deviation of all data points on the plate were computed and any data points exceeding two standard deviations of the mean plate value were considered significant. For cell viability assays, a Student's t test was carried out for each drug dose or siRNA treatment compared with no drug control or non-specific siRNA, respectively.

Identification of ROR1 as a Novel Gene Target in t(1;19)-Positive ALL

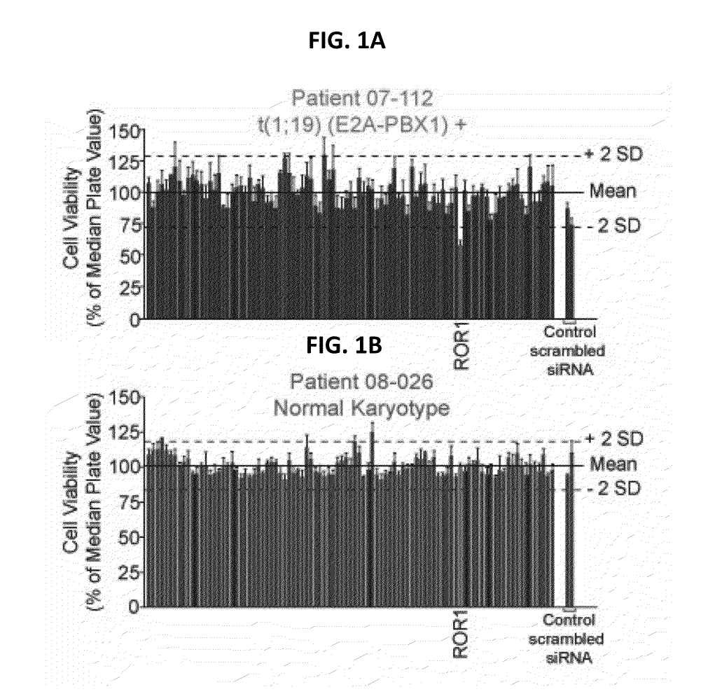

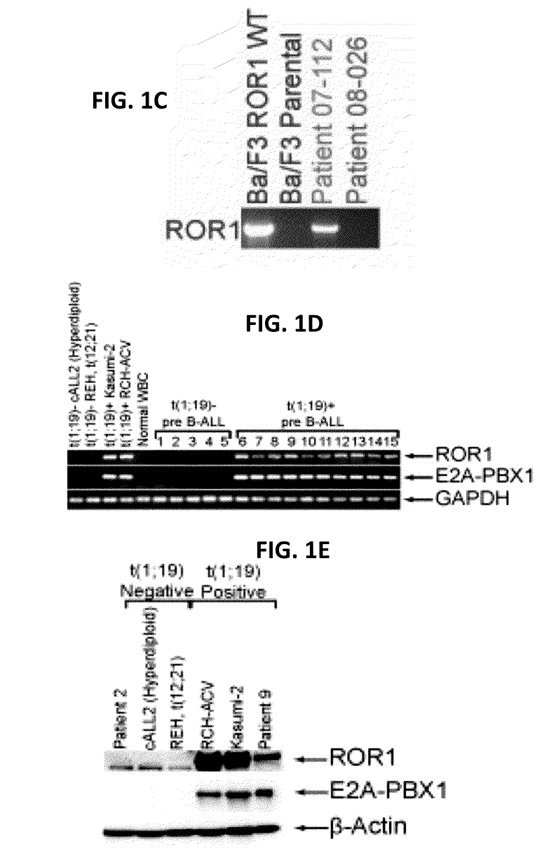

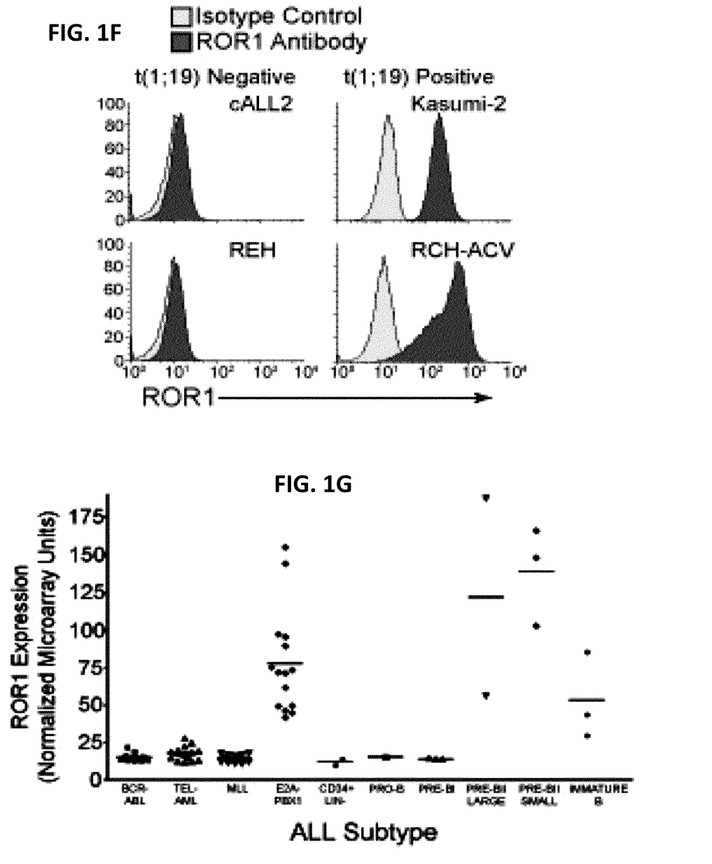

To identify new gene targets in ALL patients, clinical specimens from pediatric ALL patients were tested by gene-silencing with an siRNA library. Cells were electroporated with pre-validated siRNAs that individually target each tyrosine kinase or pseudokinase as well as non-specific, control siRNA. After four days in culture, cells were subjected to an MTS assay for assessment of cell viability. Evaluation of the t(1;19)-positive sample 07-112 revealed hypersensitivity to siRNA targeting the ROR1 (FIG. 1A). Other cases with normal karyotype (sample 08-026 is used as an example), did not exhibit sensitivity to ROR1 silencing (FIG. 1B). Further evaluation by RT-PCR revealed overexpression of ROR1 in sample 07-112 at levels comparable to artificial ROR1 overexpression in Ba/F3 cells, while sample 08-026 did not exhibit detectable ROR1 expression (FIG. 1C). To test whether the ectopic expression of ROR1 observed in t(1;19) patient 07-112 was uniformly detectable in all t(1;19) ALL samples, ten pediatric ALL samples and two cell lines that are positive for t(1;19) were obtained and compared them with five pediatric ALL samples and two cell lines that are t(1;19)-negative. It was observed that all t(1;19)-positive samples exhibited overexpression of ROR1 while none of the t(1;19)-negative samples or normal white blood cells displayed the same phenotype (FIG. 1D). Overexpression of ROR1 protein was also observed by immunoblot and FACS analysis on t(1;19)-positive cells (FIGS. 1E and 1F).

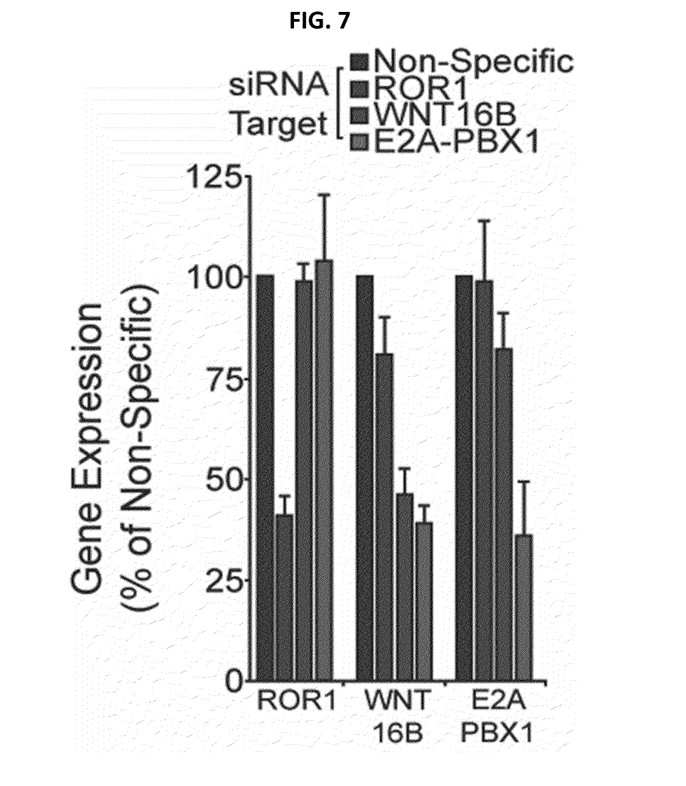

To assess the extent and exclusivity of ROR1 expression in a larger cohort of patient samples, microarray meta-analysis data generated from pediatric ALL patients and normal B-cell progenitors was examined. The t(1;19) ALL patients samples were compared with those carrying t(9;22) (BCR-ABL), t(12;21) (TEL-AML), or patients with MLL (11q23) gene rearrangements. In addition, ROR1 levels were evaluated in distinct, normal B-lineage progenitor populations (CD34⁺ Lin⁻, pro-B, pre-BI, pre-BII large, pre-BII small, and immature B cells). It was observed that there was a significantly higher level of ROR1 expression on every t(1;19) patient compared with all patients from each of the other leukemic subsets. Similarly, t(1;19) patients showed significantly higher levels of ROR1 expression compared with normal B-cell progenitor populations at the earliest stages of B-lineage development (CD34⁺ Lin⁻, proB, and pre-BI). However, when compared with normal B-lineage cells at an intermediate stage of B-cell development (large/small pre-BII and immature B), similarly high levels of ROR1 as seen on t(1;19) cells were observed (FIG. 1G). These results support recent findings showing ROR1 cell surface expression on intermediate stages of normal B-cell development. Importantly, this and other studies did not observe ROR1 expression on normal, mature B-cells and plasma cells. Hence, in non-malignant B-lineage cells, ROR1 expression appears to be absent at the earliest stages of development, becomes highly expressed at intermediate/late stages of B-lineage development, and is then downregulated in normal, mature B-cells. Interestingly, the vast majority of blasts from t(1;19) patients are arrested at this intermediate/late stage of B-lineage development (large/small pre-BII). Hence, these data demonstrate that high ROR1 expression in t(1;19) is a product of the comparatively mature differentiation state of these malignant blasts and not be due to aberrant transcription profiles of the chimeric transcription factor E2A-PBX1. Subsequent examination of the E2A-PBX1 transcription factor in t(1;19) cell lines supports this finding, as siRNA mediated knockdown of E2A-PBX1 in the t(1;19) cell line RCH-ACV showed a corresponding knockdown of the E2A-PBX1 transcriptional target WNT16B, but had no effect on expression of ROR1 (FIG. 7).

ROR1 Function is Critical for t(1;19) ALL Viability

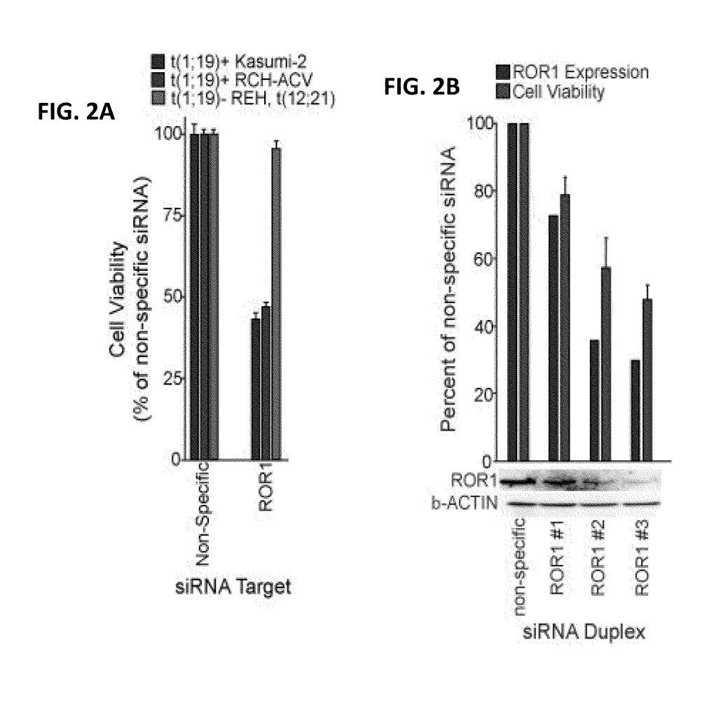

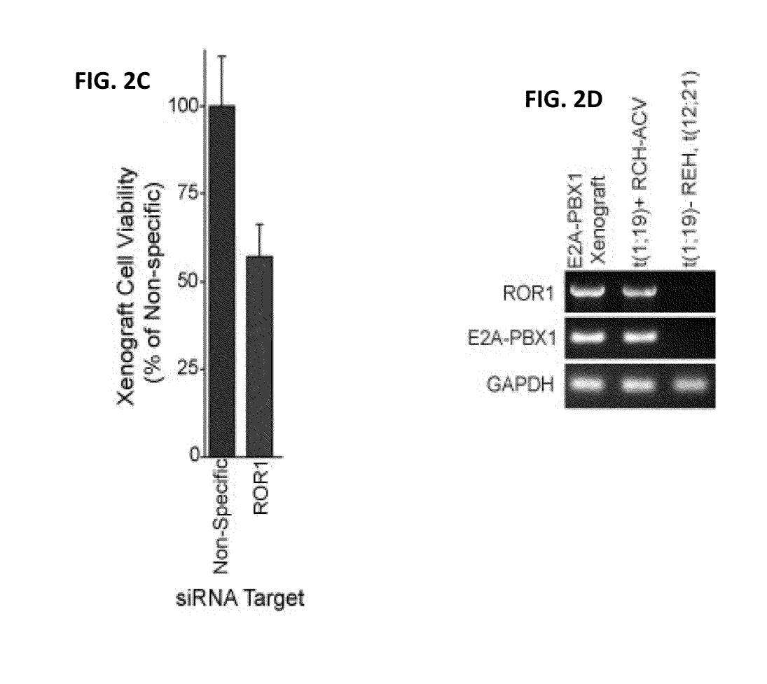

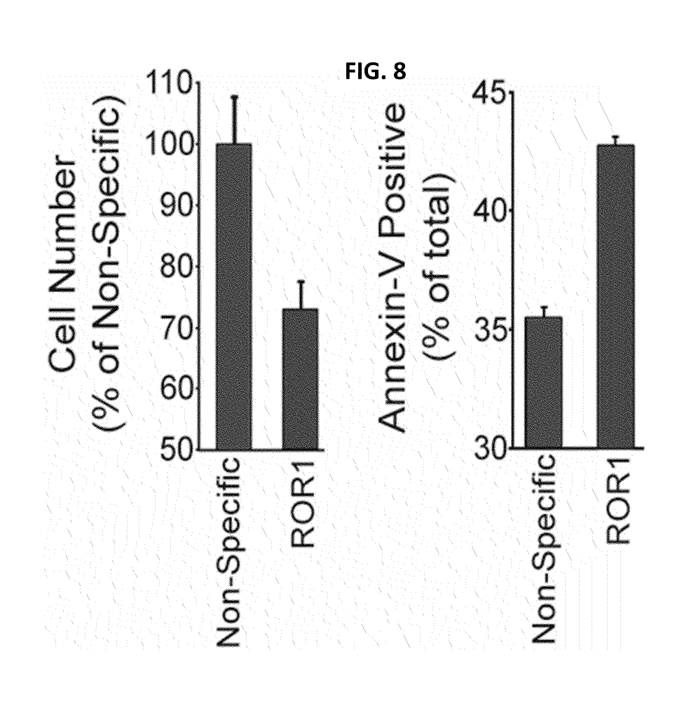

Since the two t(1;19) cell lines, RCH-ACV and Kasumi-2, recapitulated the ROR1 expression profile observed in t(1;19)-positive primary specimens, it was next tested whether these cell lines were also sensitive to ROR1 silencing, as observed with sample 07-112. Both cell lines as well as the control t(1;19)-negative cell line REH were treated with siRNA specific for ROR1. Consistent with the results from sample 07-112, both RCH-ACV and Kasumi-2 cells were sensitive to ROR1 silencing (FIG. 2A). Loss of ROR1 resulted in reduced cell growth and increased apoptosis (FIG. 8). In addition, treatment of RCH-ACV cells with 3 individual siRNA duplexes that target different portions of the ROR1 mRNA resulted in reductions of RCH-ACV cell viability that were proportional to the silencing capacity of each of the siRNA duplexes (FIG. 2B). It was next confirmed that this finding of ROR1-dependence in t(1;19) ALL samples was also reproducible in early passage t(1;19) cells propagated by xenograft into immunocompromised NOG mice. Xenograft cells derived from a t(1;19) patient were obtained and tested for ROR1 overexpression as well as for sensitivity to ROR1 siRNA. It was found that both features were recapitulated in these early passage t(1;19) xenograft cells (FIGS. 2C and 2D).

Dasatinib Impairs t(1;19) ALL Viability

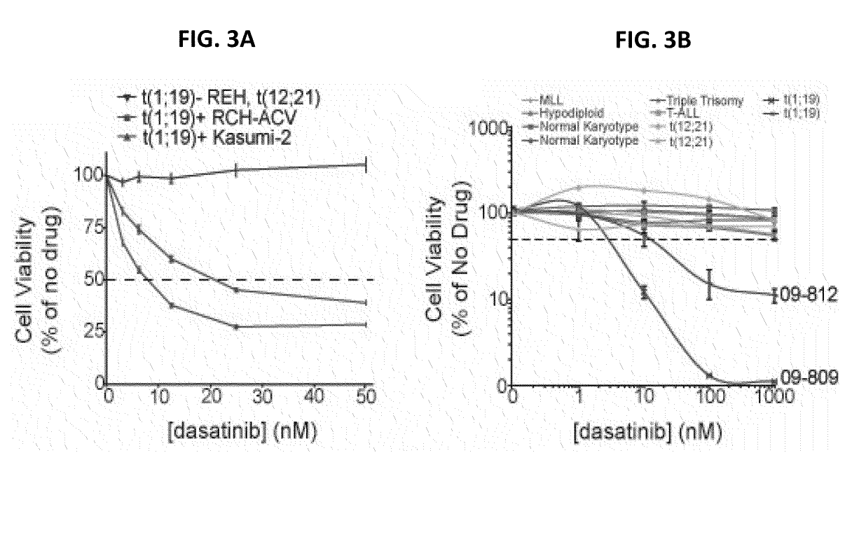

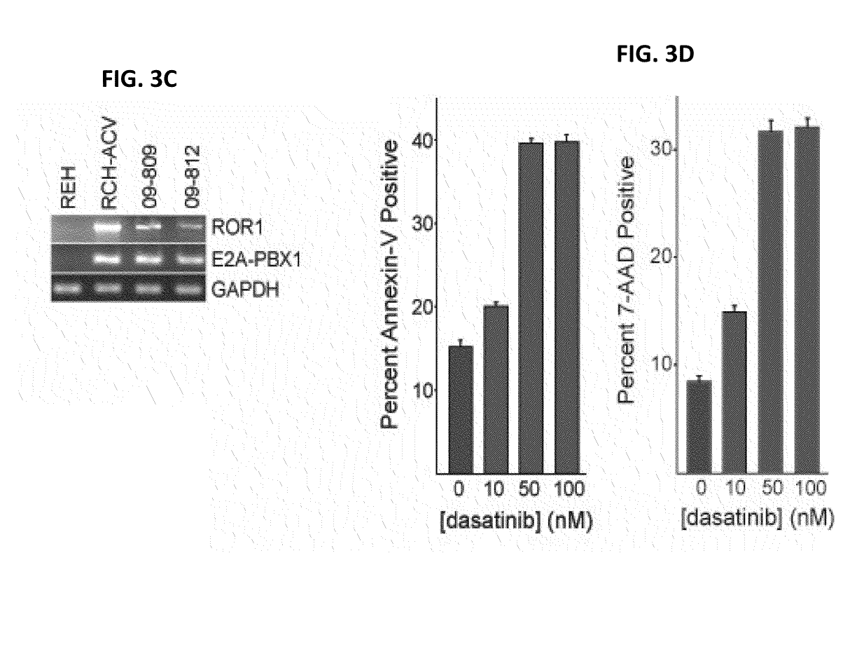

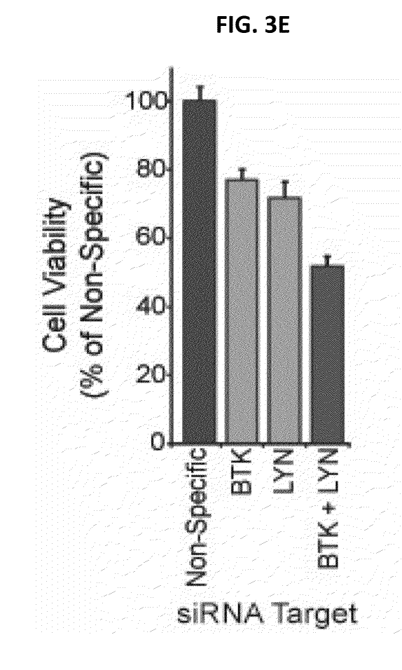

The unique and consistent overexpression of ROR1 in the t(1;19) pediatric ALL setting as well as the consistent sensitivity of t(1;19) cells to ROR1 siRNA makes ROR1 an appealing candidate target for therapeutic intervention in this subset of patients. The classification of ROR1 as similar to other cell-surface, receptor tyrosine kinase suggests several potential strategies for targeting of the ROR1 protein, including small-molecule kinase inhibitors and immunological reagents such as ROR1-specific antibodies or T-cell chimeric antigen receptors (CARs). Since small-molecule kinase inhibitors have yielded promising clinical results for a wide range of other dysregulated kinases, candidate kinase inhibitors were assessed as potential therapeutics for the t(1;19) subset of ALL. To accomplish this, a library of 66 small-molecule kinase inhibitors was employed with a broad range of target specificities. Although there is no information about the potential of these compounds for targeting of ROR1, an initial cytotoxicity screen with was conducted with two t(1;19) cell lines (RCH-ACV and Kasumi-2) as well as one ROR1-negative pediatric ALL cell line (REH) (FIGS. 1 and 2). It was observed that the FDA-approved drug dasatinib exhibited effective killing of the ROR1-positive cell lines while the ROR1-negative cell line was insensitive to dasatinib to the highest dose tested (FIG. 3A). To determine whether this observation in patient-derived cell lines was also true in primary cells taken directly from ALL patients, the effects were tested on cell viability of leukemia cells from ten ALL patients of varying disease subsets over graded concentrations of dasatinib. Two of these ten patient samples were obtained from t(1;19)-positive patients and both samples tested were highly sensitive to dasatinib, with IC₅₀ values of approximately 2 and 12 nM, while the eight other ALL samples did not achieve IC₅₀ values even at the highest tested concentration (1000 nM) (FIG. 3). Importantly, examination of ROR1 levels in these two t(1;19) patients confirmed ROR1 overexpression, similar to levels observed on all other t(1;19) samples previously tested (FIG. 3C). Exposure to dasatinib induces apoptosis in t(1;19)-positive cells, as demonstrated by annexin-V and 7-AAD staining of RCH-ACV cells post dasatinib treatment (FIG. 3D).

Dasatinib Disrupts Pre-BCR Signaling in t(1;19) ALL

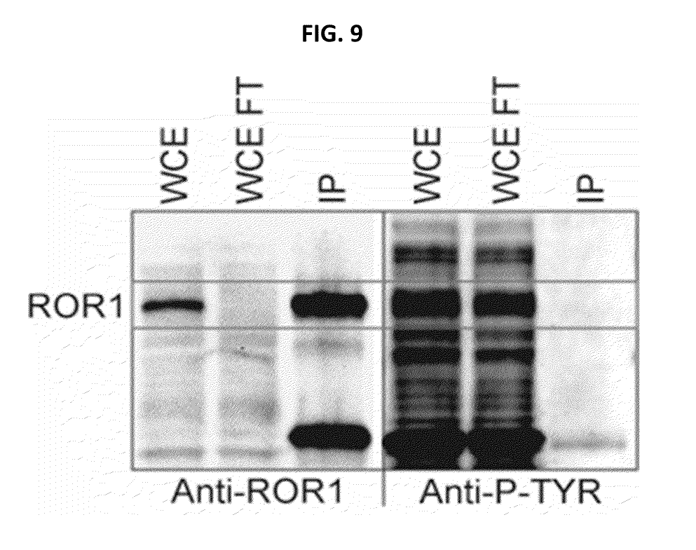

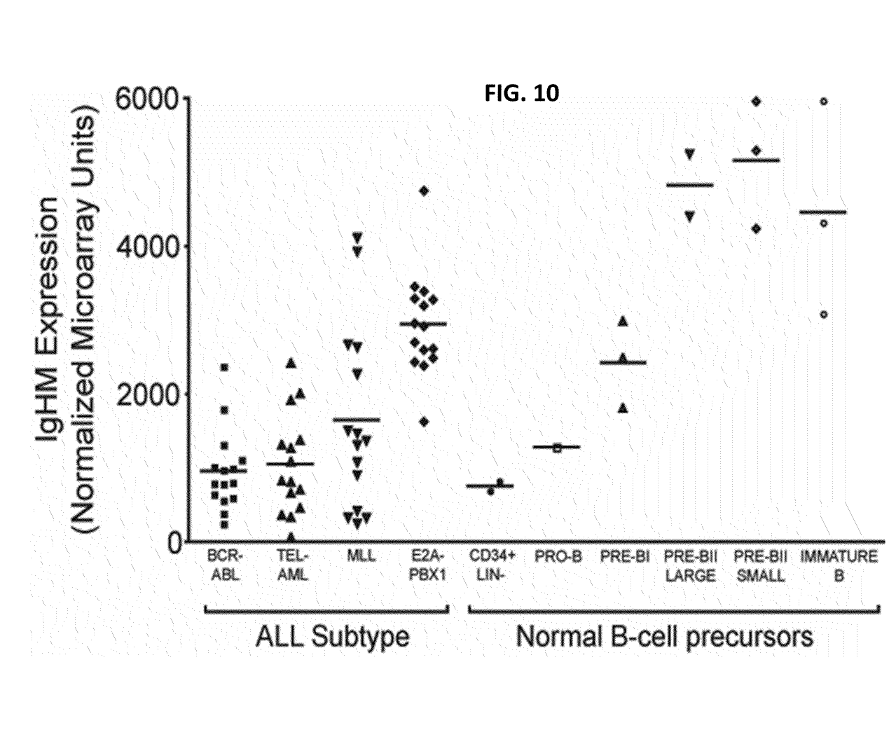

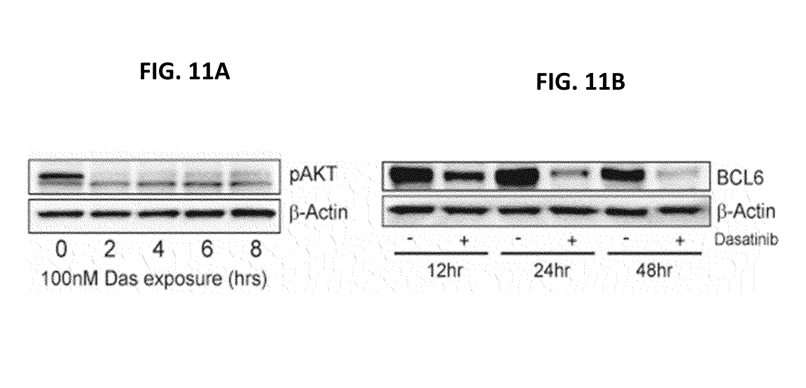

The dual sensitivity of t(1;19) ALL cells to dasatinib and ROR1 siRNA suggests that ROR1 may represent a target of dasatinib and this may mechanistically explain dasatinib sensitivity in the setting of t(1;19). However, evaluation of the phosphorylation state of ROR1 following immunoprecipitation surprisingly showed no evidence of endogenous ROR1 tyrosine phosphorylation (FIG. 9). Likewise, in vitro kinase activity assays revealed no significant ROR1 activity. Since these results would suggest that ROR1 lacks kinase function, it was theorized that alternative kinase targets must mediate dasatinib sensitivity in t(1;19) cells. As noted above, greater than 90% of t(1;19) specimens derive from cells that are arrested at the large/small pre-BII stage of B-lineage development at which stage one can also observe expression of the cytoplasmic pre-BCR complex (FIG. 10). As such, putative dasatinib targets were examined that are known to mediate pre-BCR signaling. BTK and LYN are both established targets of dasatinib and components of the pre-BCR and BCR signaling complexes. To determine whether dasatinib treatment can disrupt pre-BCR signaling, downstream signaling pathways activated or regulated by the pre-BCR were examined. One well-known downstream effector of pre-BCR signaling is the PI3K/AKT cascade. Although dasatinib does not exhibit biochemical affinity nor functional activity against PI3K or AKT, dasatinib exposure of t(1;19) cell lines show marked inhibition of AKT phosphorylation (FIG. 11A). Likewise, expression of the BCL6 transcriptional repressor, which is positively regulated by signaling from the pre-BCR, is rapidly lost in t(1;19) cell lines upon treatment with dasatinib FIG. 11B). Finally, to confirm that siRNA-mediated silencing of the dasatinib targets, BTK and LYN, could recapitulate the observed decrease of cell viability induced by dasatinib exposure in t(1;19) cells, simultaneous knockdown of both the BTK and LYN kinases in t(1;19) ALL cell lines has a significant effect on cell viability, more so than either kinase alone (FIG. 3E).

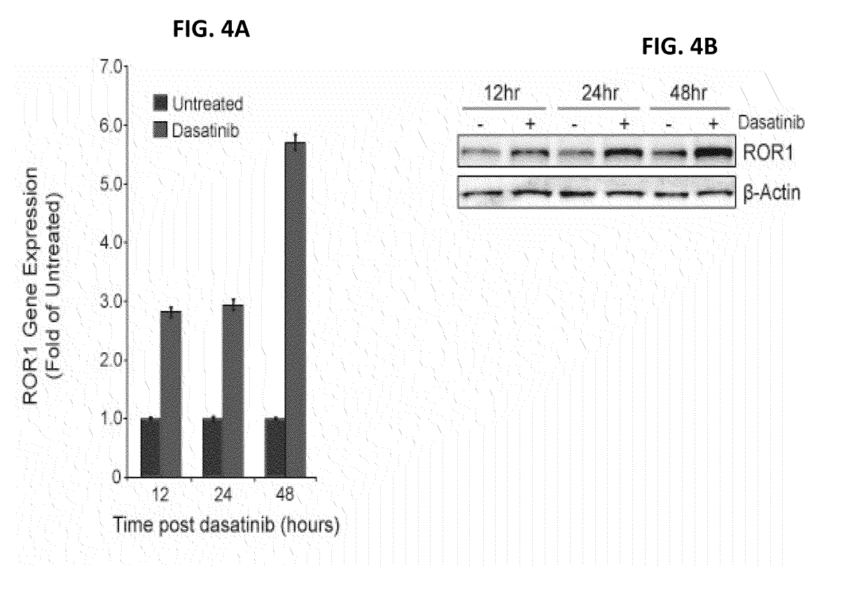

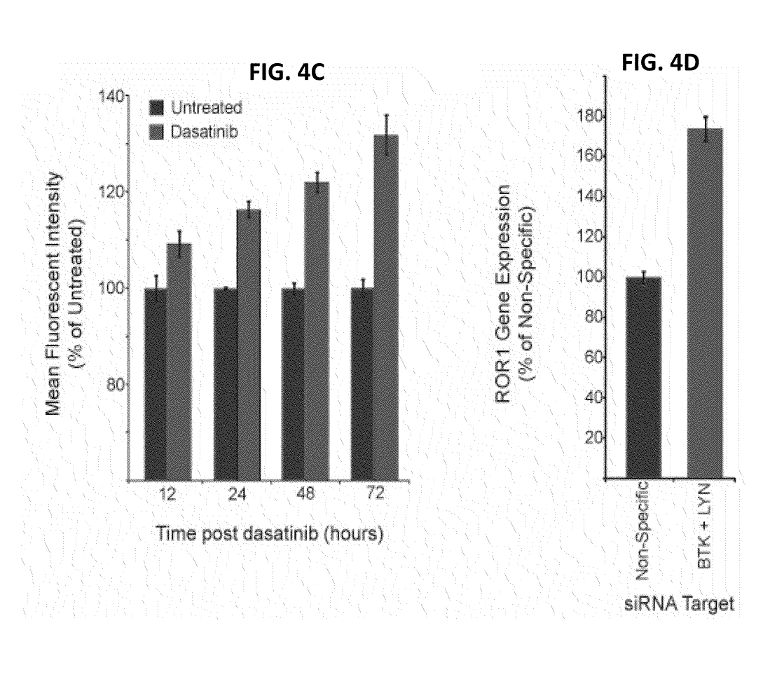

Since the upregulation of ROR1 during normal B-cell maturation correlates with assembly of and signaling from the pre-BCR complex, it was tested whether pre-BCR signaling may drive ROR1 upregulation, and ROR1 might act as an effector of pre-BCR induced protection against apoptosis. To test this hypothesis, it was examined whether dasatinib inhibition of the pre-BCR (via inhibition of BTK/LYN) would result in decreased ROR1 expression. Thus, ROR1 expression levels were examined after dasatinib treatment. Paradoxically, treatment of t(1;19) ALL cell lines with dasatinib resulted in rapid increases of ROR1 mRNA, protein, and cell surface expression (FIG. 4A-4C). Further, disruption of the pre-BCR by silencing of both BTK and LYN also resulted in upregulation of ROR1, confirming that ROR1 upregulation induced by dasatinib is mediated by this same pre-BCR signaling complex (FIG. 4D). Together, these results show dasatinib inhibits BTK and LYN kinase and disrupts signaling from the pre-BCR. Further, impaired pre-BCR signaling leads to upregulation of ROR1.

### ROR1 Dependence and Dasatinib Sensitivity in B-cell Malignancies

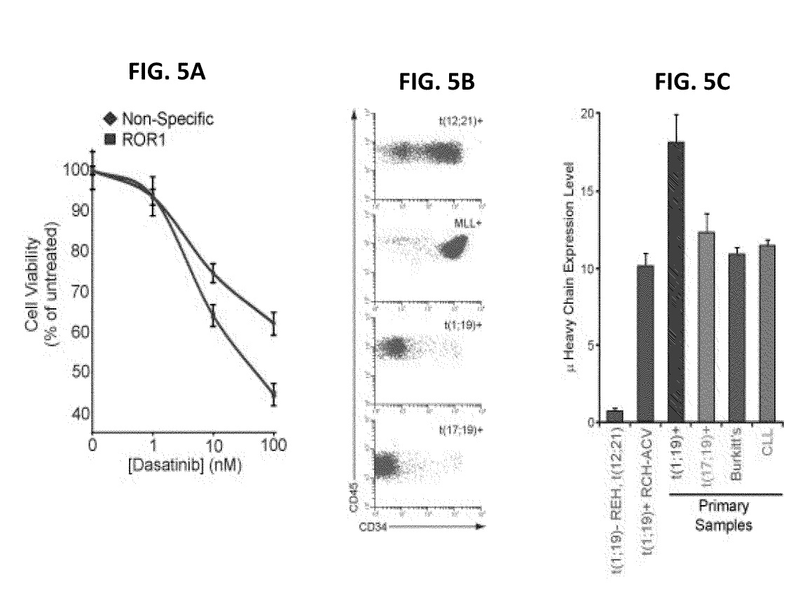

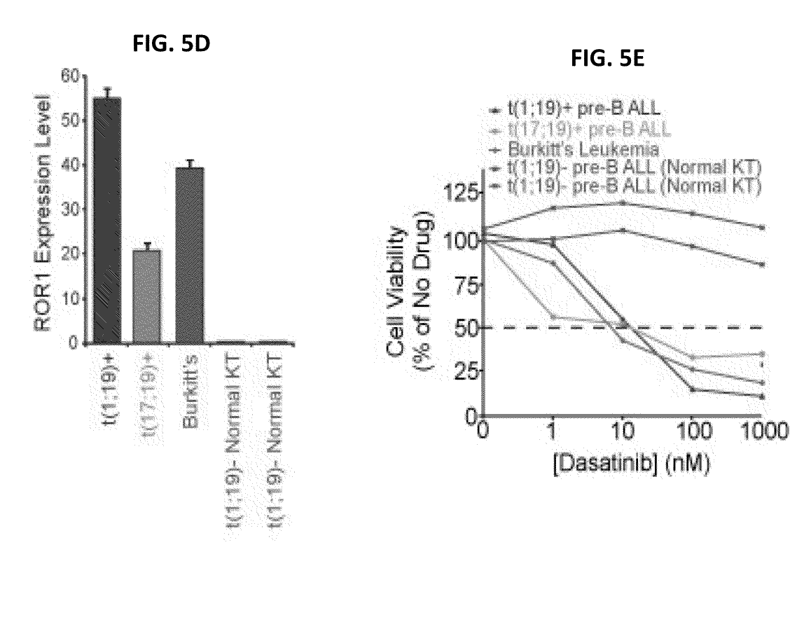

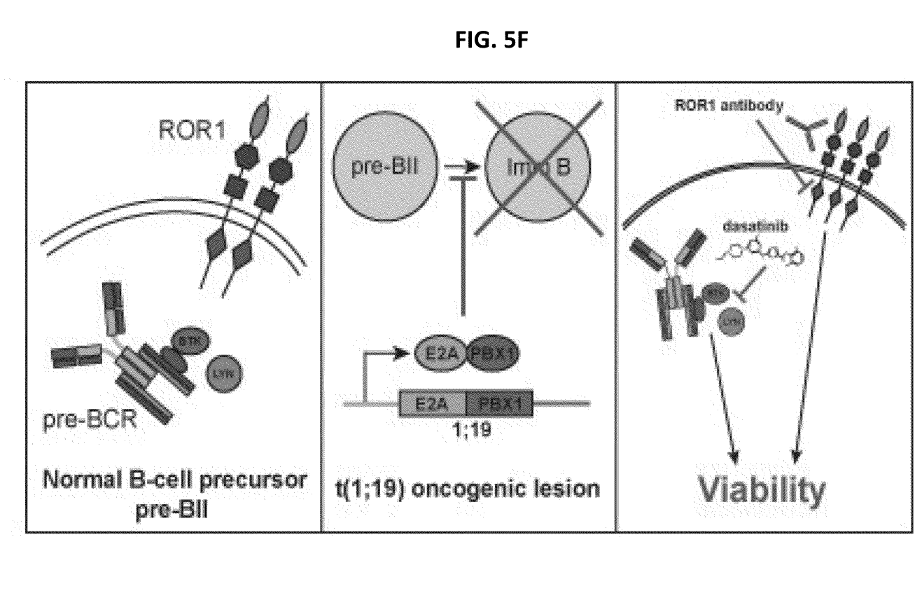

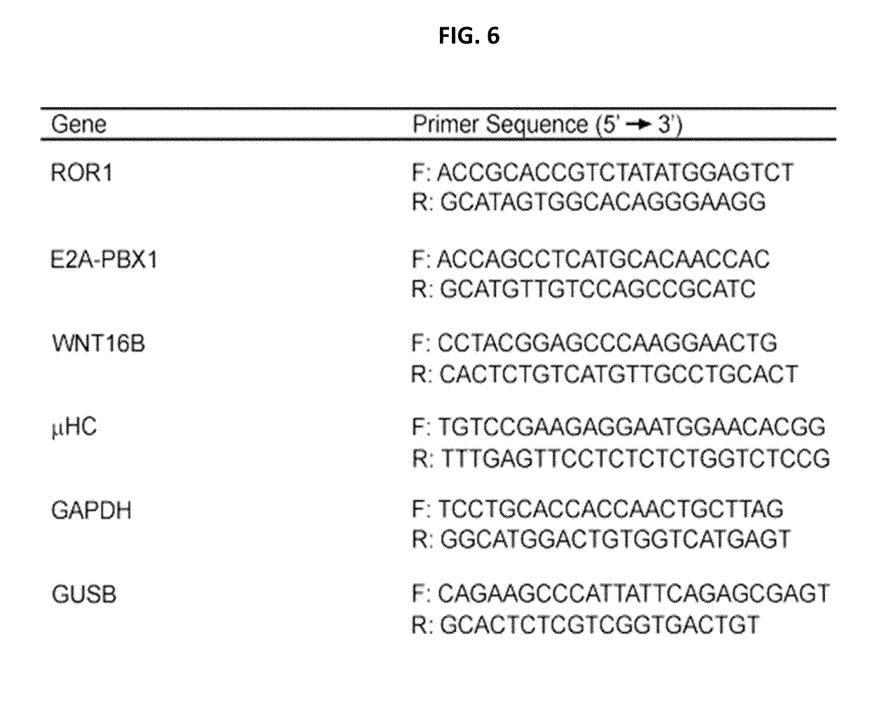

Upregulation of ROR1 in response to pre-BCR inhibition by dasatinib may represent a cell survival rescue pathway. If this were the case, then targeting of ROR1 in the context of dasatinib exposure would potentiate dasatinib effects on t(1;19) cells. To test this hypothesis, t(1;19) ALL cell lines were treated with ROR1 or non-specific siRNA and subsequently exposed to graded concentrations of dasatinib. As predicted, cells subjected to ROR1 silencing were more sensitive to dasatinib compared with cells treated with non-specific siRNA (FIG. 5A). This additive effect indicates that ROR1, functions as a rescue pathway for cell survival in the context of pre-BCR inhibition, and mitigation of this rescue pathway by alternate means (ROR1 siRNA) can further abrogate cell viability. It also demonstrates the benefit of therapeutic strategies that would simultaneously target both ROR1 (with RNAi or other means) and the pre-BCR (with dasatinib).

Finally, since t(1;19) ALL does not represent the only B-lineage malignancy that is arrested at an intermediate or mature stage of B-cell development, ROR1 status and dasatinib sensitivity was analyzed in additional B-cell malignancies from intermediate or mature stages of B-cell development. Primary samples were examined from patients diagnosed with the intermediate/mature B-cell malignancies, t(17;19) ALL and Burkitt's leukemia, both of which are CD34-negative and express either the pre-BCR or mature-BCR as indicated by μ heavy chain (μHC) expression (FIGS. 5B and 5C). Both the t(17;19) ALL and Burkitt's samples showed high ROR1 expression levels, similar to those observed in t(1;19) ALL samples (FIG. 5D). As expected, these samples were also sensitive to dasatinib treatment, with IC50s comparable to t(1;19) ALL (FIG. 5E). These results demonstrate that ROR1 expression is a conserved characteristic of intermediate/mature B-cell malignancies and dasatinib treatment represents a viable therapeutic option for additional B-lineage malignancies that express the pre- or mature-BCR.

Taken together, the results presented herein identify a model in which the 1;19 translocation arrests normal B-cell development at a stage in which ROR1 is naturally upregulated and pre-BCR signaling is critical for cell viability (FIG. 5F). Although ROR1 and the pre-BCR complex are not mutated oncogenes, they do represent onco-requisite pathways and so remain viable therapeutic targets for the treatment of t(1;19) ALL. The observation of ROR1 overexpression and dasatinib sensitivity in additional B-cell malignancies indicates this paradigm is more broadly applicable to B-lineage malignancies that are arrested at an intermediate or mature stage of B-cell development.

Using an siRNA-based functional screen, disclosed herein is a new gene target for potential therapeutic intervention in t(1;19) ALL patients. As demonstrated herein this receptor tyrosine kinase, ROR1, is consistently expressed on all t(1;19) samples and that its presence is required for t(1;19) ALL viability. These findings offer new insight into the functional importance of ROR1 in t(1;19) ALL as well as in the normal B-cell maturation process, yet little is known about the biological role of ROR1 in these settings. It is shown that ROR1 upregulation in t(1;19) ALL is a product of B-lineage development arrest at the pre-BII stage of B-cell development and not a result of aberrant regulation by the E2A-PBX1 transcription factor generated by the 1;19 translocation. These studies also show ROR1 is upregulated in t(17;19) ALL and Burkitt's leukemia/lymphoma, indicating that ROR1 may be expressed in most B-cell malignancies arrested at an intermediate/mature stage of development. Importantly, the expression of ROR1 on cells from the mature B-lineage malignancies, CLL, MCL and Burkitt's, demonstrates an important deviation from the normal distribution of ROR1-expression observed in B-cell development. While ROR1-expression is observed only in the intermediate stage of normal B-cell development³², it is observed in both intermediate and mature B-cell malignancies. This important distinction suggests that retention of ROR1-expression into the mature stages of B-cell maturation plays a role in maintenance of cell viability of these mature, malignant B-cell clones. The specific molecular interactions that regulate expression from the ROR1 locus and its biochemical function within these cells are unresolved questions that must still be addressed.

This work identifies ROR1 as a consistent therapeutic target in t(1;19) ALL and other B-cell malignancies. Immunological based therapies present a promising therapeutic strategy for specifically targeting ROR1-positive malignancies. Immunotherapies that utilize chimeric antigen receptor (CAR)-modified T-cells targeting B-cell lineage-specific surface markers, such as CD19 and CD20, are actively being investigated in clinical trials for B-cell malignancies. The results presented herein indicate that a similar strategy targeting ROR1 could prove valuable in treating most intermediate/mature B-cell malignancies.

Small molecule kinase inhibitors have proven clinically successful and present a more immediate clinical option for these B-cell malignancies. The studies described herein show dasatinib impairs the viability of t(1;19) ALL primary samples and cell lines at clinically relevant concentrations and that the primary dasatinib targets in this setting are kinases associated with the pre-BCR signaling complex, BTK and LYN. This explains the lack of sensitivity to dasatinib in the pre-B-cell line REH and other primary ALL samples that lack expression of the pre-BCR due to differentiation arrest at earlier stages of B-lineage development where the pre-BCR complex is not yet expressed. Conversely, as disclosed herein, high ROR1 expression and dasatinib-sensitivity in primary t(17;19) ALL and Burkitt's lymphoma samples due to developmental arrest at a comparatively mature stage of B-cell differentiation in which the malignant cells do express either the pre- or mature-BCR.

### Example 2

### Mouse Model of Treatment with a ROR1 Kinase Inhibitor

This example describes exemplary procedures for testing the efficacy of treatment with an inhibitor of ROR1 signaling activity.

Candidate agents that have been identified as most potently reducing the viability of ROR1-sensitive but not ROR1-resistant cell lines and patient samples, are evaluated in vivo, in a xenograft model of ALL in immunocompromised mice, using both cell lines and ALL patient samples. ROR1-dependent cells are transplanted mice and the mice are treated with candidate ROR1 inhibitors. The mice are administered a therapeutic amount of candidate agent identified as one inhibiting ROR1 signaling activity. The candidate agent can be administered at doses of 0.0001 μg/kg body weight to about 10 mg/kg body weight per dose, such as 0.0001 μg/kg body weight-0.001 μg/kg body weight per dose, 0.001 μg/kg body weight-0.01 μg/kg body weight per dose, 0.01 μg/kg body weight-0.1 μg/kg body weight per dose, 0.1 μg/kg body weight-10 μg/kg body weight per dose, 1 μg/kg body weight-100 μg/kg body weight per dose, 100 μg/kg body weight-500 μg/kg body weight per dose, 500 μg/kg body weight per dose-1000 μg/kg body weight per dose, 1.0 mg/kg body weight per dose-10 mg/kg body weight per dose or even greater. However, the particular dose can be determined by a skilled clinician. The candidate agent can be administered in several doses, for example continuously, daily, weekly, or monthly.

The mode of administration can be any used in the art. The amount of agent administered to the subject can be determined by a clinician, and may depend on the particular subject treated. Specific exemplary amounts are provided herein (but the disclosure is not limited to such doses). Mice will be monitored over a time-course for evidence of ALL cell growth. At the completion of the trial, all mice will be sacrificed and subjected to complete necropsy. Multiple organs, including spleen, bone marrow, peripheral blood, liver, and lung, are evaluated for involvement of neoplastic cells.

### Example 3

### Treatment of Subjects

This example describes methods that can be used to treat a subject having a particular disease or condition, such as B-ALL that can be treated by an identified inhibitor of ROR1 signaling activity. Such a therapy can be used alone, or in combination with other therapies (such as the administration of a chemotherapeutic agent).

In particular examples, the method includes screening a subject having or thought to have a particular disease or condition treatable by an inhibitor of ROR1 signaling activity. Subjects of an unknown disease status or condition can be examined to determine if they have a disease or condition treatable by an inhibitor of ROR1 signaling activity for example by using the methods described herein.

The subject can be administered a therapeutic amount of an inhibitor of ROR1 signaling activity. The inhibitor of ROR1 signaling activity can be administered at doses of 0.0001 μg/kg body weight to about 10 mg/kg body weight per dose, such as 0.0001 μg/kg body weight-0.001 μg/kg body weight per dose, 0.001 μg/kg body weight-0.01 μg/kg body weight per dose, 0.01 μg/kg body weight-0.1 μg/kg body weight per dose, 0.1 μg/kg body weight-10 μg/kg body weight per dose, 1 μg/kg body weight-100 μg/kg body weight per dose, 100 μg/kg body weight-500 μg/kg body weight per dose, 500 μg/kg body weight per dose-1000 μg/kg body weight per dose, or 1.0 mg/kg body weight per dose-10 mg/kg body weight per dose. However, the particular dose can be determined by a skilled clinician. The inhibitor of ROR1 signaling activity can be administered in several doses, for example continuously, daily, weekly, or monthly. The administration can concurrent or sequential.

The mode of administration can be any used in the art. The amount administered to the subject can be determined by a clinician, and may depend on the particular subject treated. Specific exemplary amounts are provided herein (but the disclosure is not limited to such doses).

A ten percent reduction in one or more sign or symptoms associated with the disease or condition associated with aberrant ROR1 signaling activity indicates that the treatment is effective.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for selecting a subject suspected of having or having ROR1-dependent lymphoblastic leukemia for treatment with an agent that inhibits ROR1, BTK and/or LYN signaling activity, comprising:
detecting ROR1 overexpression or aberrant signaling activity of ROR1 in a white blood cell or bone marrow cell obtained from the subject, wherein detecting ROR1 overexpression or aberrant signaling activity of ROR1 identifies the subject to be selected for treatment with an agent that inhibits ROR1, BTK and/or LYN signaling activity.

2. The method of claim 1, wherein detecting aberrant signaling activity of ROR1 comprises:
contacting a white blood cell or bone marrow cell obtained from the subject with an agent that inhibits ROR1 signaling activity; and
detecting a ROR1-regulated signaling activity, wherein an alteration in the ROR1-regulated signaling activity in the white blood cell or bone marrow cell as compared to a control identifies the subject to be selected for treatment with an agent that inhibits ROR1, BTK and/or LYN signaling activity.

3. The method of claim 2, wherein the ROR1-regulated signaling activity is one or both of cell proliferation and cell viability of the white blood cell or bone marrow cell, and wherein the alteration is a decrease in one or both of cell proliferation and cell viability of the white blood cell or bone marrow cell as compared to the control and identifies the subject to be selected for treatment with an agent that inhibits ROR1, BTK and/or LYN signaling activity.

4. The method of a claim 3, wherein detecting proliferation of the cell or viability of the cells comprises performing a 3-(4,5-Dimethylthiazol-2-yl)-5-(3-Carboxymethoxyphenyl)-2-(4-Sulfophenyl)-2H-Tetrazolium (MTS) assay, wherein the MTS assay comprises contacting the white blood cell or the bone marrow cell with MTS and measuring the formation of formazan.

5. The method of claim 2, wherein the ROR1-regulated signaling activity is the phosphorylation of components in the biological signaling pathway comprising ROR1, and wherein the alteration is a decrease phosphorylation of a downstream target in the biological signaling pathway comprising ROR1 and identifies the subject to be selected for treatment with an agent that inhibits ROR1, BTK and/or LYN signaling activity.

6. The method of claim 2, wherein the ROR1-regulated signaling activity is the phosphorylation of components in the biological signaling pathway comprising ROR1, and wherein the alteration is an increase phosphorylation of a downstream target in the biological signaling pathway comprising ROR1 and identifies the subject to be selected for treatment with an agent that inhibits ROR1, BTK and/or LYN signaling activity.

7. The method of claim 2, wherein the agent that regulates ROR1 signaling activity comprises a small molecule inhibitor of ROR1 signaling activity or an inhibitory RNA that specifically targets ROR1.

8. The method of claim 1, wherein detecting ROR1 overexpression, comprises:
detecting an amount of ROR1 expressed in a white blood cell or bone marrow cell obtained from the subject;
comparing the amount of ROR1 expressed in a white blood cell or bone marrow cell to a control,
wherein an increase in the amount of ROR1 expressed in a white blood cell or bone marrow cell relative to the control identifies the subject to be selected for treatment with an agent that inhibits ROR1, BTK and/or LYN signaling activity.

9. The method of claim 8, wherein the control is a standard reference value for basal levels of ROR1 expression.

10. The method of claim 8, wherein the control is an amount of ROR1 expression in a biological sample obtained from a subject that does not have a ROR1-dependent leukemia.

11. The method of claim 9, wherein the control is an amount of ROR1 expression in a biological sample obtained from a subject that does not have a ROR1-dependent leukemia.

12. The method of claim 8, wherein detecting the amount of ROR1 expressed comprises detecting an amount of ROR1 RNA or detecting an amount ROR1 protein.

13. The method of claim 12, wherein detecting the amount of ROR1 protein comprises the use of an immunohistochemical assay, a radioimmunoassay, a Western blot assay, an immunofluorescent assay, an enzyme immunoassay, a chemiluminescent assay, florescent activated cell sorting, mass spectrometry assay, or any combination thereof.

14. The method of claim 12, wherein detecting the amount of ROR1 protein comprises, the use of a specific binding agent that specifically binds ROR1.

15. The method of claim 14, wherein the specific binding agent is detectably labeled.

16. The method of claim 14, wherein the specific binding agent is an antibody.

17. The method of claim 12, wherein determining the amount of ROR1 RNA comprises probe hybridization, nucleic acid amplification or any combination thereof.

18. The method of claim 1, wherein the method is a method of diagnosing the subject as having a ROR1-dependent leukemia.

19. The method of claim 1, further comprising administering to the subject a small molecule inhibitor of ROR1, BTK and/or LYN signaling activity.

20. The method of claim 19, wherein the small molecule inhibitor of BTK and/or LYN signaling activity comprises dasatinib.

21. The method of claim 1, wherein the subject is a human subject.

22. The method of claim 1, wherein the subject is diagnosed as having t(1;19) ALL, t(17;19) ALL, or Burkitt's lymphoma.

23. The method of claim 1, further comprising selecting the subject for treatment with an agent that inhibits ROR1, BTK and/or LYN signaling activity

24. A method for identifying an agent of use in treating a subject with a ROR1-dependent leukemia or with a predisposition for developing a ROR1-dependent leukemia, comprising;
contacting an isolated test cell that is dependent on ROR1 signaling activity with a test agent;
detecting a ROR1-regulated signaling activity; and
comparing the amount of the ROR1-regulated signaling activity in the test cell to a control, wherein an alteration in the ROR1-regulated signaling activity in the test cell relative to the control indicates that the agent is useful for the treatment of a subject with ROR1-dependent leukemia or with a predisposition for developing a ROR1-dependent leukemia.

25. The method of claim 24, wherein the ROR1-regulated signaling activity is one or both of cell proliferation and cell viability of the test cell, and wherein the alteration is a decrease in one or both of cell proliferation and cell viability of test cell as compared to a control and identifies the agent as useful for the treatment of a subject with ROR1-dependent leukemia or with a predisposition for developing a ROR1-dependent leukemia.

26. The method of claim 25, wherein detecting proliferation or viability comprises performing a 3-(4,5-Dimethylthiazol-2-yl)-5-(3-Carboxymethoxyphenyl)-2-(4-Sulfophenyl)-2H-Tetrazolium (MTS) assay, wherein the MTS assay comprises contacting the test cell with MTS and measuring the formation of formazan.

27. The method of claim 24, wherein the ROR1-regulated signaling activity is the phosphorylation of components in the biological signaling pathway comprising ROR1, and wherein the alteration is a decrease phosphorylation of a downstream target in the biological signaling pathway comprising ROR1 and identifies the agent as useful for the treatment of a subject with ROR1-dependent leukemia or with a predisposition for developing a ROR1-dependent leukemia.

28. The method of claim 24, wherein the ROR1-regulated signaling activity is the phosphorylation of components in the biological signaling pathway comprising ROR1, and wherein the alteration is an increase phosphorylation of a downstream target in the biological signaling pathway comprising ROR1 and identifies the agent as useful for the treatment of a subject with ROR1-dependent leukemia or with a predisposition for developing a ROR1-dependent leukemia.

29. The method of claim 24, wherein the control is a standard value or the amount of ROR1 signaling activity of a second isolated test cell that is not dependent on ROR1 signaling activity that is contacted with the test agent or a control cell that is not dependent on ROR1 signaling activity.

30. The method of claim 24, wherein the agent that regulates ROR1 signaling activity is a small molecule inhibitor of ROR1 signaling activity or an inhibitory RNA that specifically targets ROR1.

31. The method of claim 30, wherein the inhibitory RNA that specifically targets ROR1 is a siRNA, microRNA, shRNA, or ribozyme.

32. A method for treating or inhibiting a ROR1-dependent leukemia in a subject, comprising:
selecting a subject with ROR1-dependent leukemia; and
administering to the subject a therapeutically effective amount of an inhibitor of ROR1, BTK and/or LYN signaling activity, thereby treating or inhibiting ROR1-dependent leukemia in the subject.

33. The method of claim 32, wherein the ROR1-dependent leukemia is acute lymphoblast leukemia (ALL).

34. The method of claim 33, wherein the ALL is B-ALL.

35. The method of claim 34, wherein the B-ALL is pediatric B-ALL.

36. The method of claim 32, wherein the subject is a human subject.

37. The method of claim 32, wherein the inhibitor of ROR1 signaling activity is a small molecule inhibitor of ROR1 signaling activity or an inhibitory RNA that specifically targets ROR1.

38. The method of claim 36, wherein the inhibitory RNA that specifically targets ROR1 is a siRNA, microRNA, shRNA, or ribozyme.

39. The method of claim 32, wherein the subject is diagnosed as having t(1;19) ALL, t(17;19) ALL, or Burkitt's lymphoma.
